(19) United States
(12) Patent Application Publication
(10) Pub. No.: US 2019/0093096 A1
(43) Pub. Date: Mar. 28, 2019

(54) COMOSAIN AND BROMELAINS AS IMMUNO-THERAPY IN THE TREATING AND/OR PREVENTING VARIOUS TYPES OF CANCER IN A MAMMAL

(71) Applicant: BENEDICT SCHUE LIAO, WEST COVINA, LOS ANGELES, CA (US)

(72) Inventor: BENEDICT SCHUE LIAO, LOS ANGELES, CA (US)

(73) Assignees: ALEX BISMARK LIAO, LOS ANGELES, CA (US); AUSTIN LEWIS LIAO, LOS ANGELES, CA (US); FU-CHUAN JUDY LI-LIAO, LOS ANGELES, CA (US); BURTON ARTHUR LIAO, LOS ANGELES, CA (US)

(21) Appl. No.: 15/731,991

(22) PCT Filed: Sep. 2, 2017
(86) PCT No.: PCT/US17/00053

§ 371 Date: Jul. 19, 2018

Related U.S. Application Data
(60) Provisional application No. 62/602,080, filed on Apr. 10, 2017.

(51) Int. Cl. C12N 9/50; A61P 35/00

(52) U.S. Cl. CPC C12N 9/63; A61K 38/00; A61P 35/00; C12Y 304/22032

(57) ABSTRACT

The Cloning and Expression of Comosain's Minigene and the Methods and Compositions for treating and/or preventing various types of cancer which comprise administered an effective amount of Glyco-polypeptides and Bioflavonoids such as Comosain, Ananase, Bromelainases, Trypsin, Pepsin, Quercetin, Rutin, Naringenin, Genistein, Hesperetin, etc and/or a mixture thereof.

We combine this patent application with previous application of US/2012/00323 (application Ser. No. 13/374,328, filing date on Dec. 16, 2011) and WO/2013/089803 as a combined entity. Previous invention title as “Bromelainases ((Comosain)) as Chemotherapeutic agents in treating and or preventing various types of cancer in a mammal”. Copy of this certificate also herein enclose for your reference and expertise consideration.

REFERENCES CITED: (U.S. Patent Documents)

| Pat. No. | Date | Inventors |
|---|---|---|
| US/2012/000323 | June 2012 | Ben. Liao, Alex Liao et al |
| U.S. Pat. No. 3,002,891 | October 1961 | Heinicke et al |
| U.S. Pat. No. 2,950,227 | August 1960 | Gibian, & Bratfisch et al |
| U.S. Pat. No. 6,835,809 | December 2004 | Liu, et al |
| U.S. Pat. No. 3,691,016 | September 1972 | Patel, et al |
| U.S. Pat. No. 4,247,642 | January 1981 | Hirohara, et al |
| U.S. Pat. No. 5,580,755 | December 1996 | Souza, et al |
| U.S. Pat. No. 5,582,823 | December 1996 | Souza, et al |
| U.S. Pat. No. 6,835,809 | December 2004 | Liu, and Feige et al |
| U.S. Pat. No. 7,335,382 | February 2008 | Ding, and Adrian et al |
| 6,180,660 | January 2001 | Whitney et al. |
| 6,238,673 | May 2001 | Howard et al. |
| 20010046963 | November 2001 | Wenzel et al. |
| 6,225,338 | May 2001 | Romanczyk, et al. |
| 6,221,357 | April 2001 | Bok, et al. |
| 4,346,227 | August 1982 | Terehara, et al. |
| 5,354,772 | October 1994 | Kathawala, et al. |
| W0/2013/089803 | June 2013 | Liao, B. Alex Liao et al. |
| U.S. Pat. No. 2,421,061 | 1947 | Higby, Ca. Fruit Growers |
| | | Exchange |
| U.S. Pat. No. 2,421,062 | 1947 | Pulley, Von Loesecke, Ca. |
| | | Fruit Growers Exchange |
| U.S. Pat. No. 2,421,063 | 1947 | Pulley, Von Loesecke, Ca. |
| | | Fruit Growers Exchange |
| U.S. Pat. No. 2,442,110 | 1948 | Baier, Ca. Fruit Growers |
| | | Exchange |

### OTHER REFERENCES

1. Taussig S J.; and Batkin S.; et al: Bromelain, the enzyme complex of ananas comosus. Journal of Ethno pharmacology 22: pg. 191-203, (1988)
2. Taussig S.; Batkin S.; and Szekerczes J.; et al: Antimetastatic effect of Bromelain with or without its proteolytic and anticoagulant activity. Journal Cancer Research Clinical Oncology 114; pg. 507-508, (1988).
3. Taussig S J.; Szekerczes J; and Batkin S. et al: Inhibition of tumor growth in vitro by Bromelain, an extract of pineapple plant (ananas comosus Planta Medicus 6; pg. 538-539, (1985).
4. Liao, Benedict; Liao, Alex; Liao, Austin; Liao, Judy; Liao, Burton; Liao, Justin; Liao, Edward; Liao, Robert; Liao, Lily; et al; Bromelainanses (Bromelain proteinases) as chemotherapy agents in treating and/or preventing various types of cancer in the mammal; US Patent US/2012/000323, (via PCT, Patent Cooperation Treaty) and International Patent WO/2013/089803
5. Liao, Benedict; Liao, Alex; Liao, Austin; Liao, Judy; Liao, Burton; Liao, Justin; Liao, Edward; Liao, Robert; Liao, Lily; et al; a method for treating and/or preventing the cardiovascular and hepatic diseases induced by hyperlipidemia which comprises administered thereto an effective amount of bioflavonoids extract derived from fructus crataegus such as rutin, quercetin, kaempferol and vitexin, or a mixture thereof. (2003, Provisional Patent, published June. 2004)
6. Taussig S J.; and Batkin S.; et al: Bromelain, the enzyme complex of ananas comosus. Journal of Ethno pharmacology 22: pg. 191-203, (1988).
7. Taussig S.; Batkin S.; and Szekerczes J.; et al: Antimetastatic effect of Bromelain with or without its proteolytic and anticoagulant activity. Journal Cancer Research Clinical Oncology 114; pg. 507-508, (1988).
8. Taussig S J.; Szekerczes J; and Batkin S. et al: Inhibition of tumor growth in vitro by Bromelain, an extract of pineapple plant (ananas comosus), Planta Medicus 6; pg. 538-539, (1985).
9. Maurer H R.; Hozumi M.; Honma Y.; and Okabe-Kado J. et al.; Bromelain induces the differentiation of leukemic cells in vitro; an explanation for its cytostatic effect Planta medicus 54; pg. 377-381, (1988).
10. Saija A., Scalese M. et al.: Plant and/or fruit flavonoids such as Rutin, Quercetin, Naringenin and Hesperetin in inhibiting tumor growth (Free Radical Biology and Medicine vol. 19, no. 4 pg. 481-486, (1995)
11. Felicia, V.S.; Najla Guthrie, et al: Inhibition of breast cancer cell proliferation and delay of mammary tumor-genesis by flavonoids and citrus juices. (Nutrition and Cancer vol. 26, no.2, pg. 167-181, (1996).
9. Revilla, E., and Ryan J. M. et al.; analysis of several phenolic compounds with potential antioxidant properties in grape extracts and wines by high-performance liquid chromatography-photodiode array detection without sample preparation. (Journal of Chromotographia; 6, 881 (1-2); pg. 461-469, (2000).
10. Rowan A. et al: Pineapple cysteine endopeptidases, Methodology Enzymol. (Vol. 244: pg. 555-568 (1994).
11. Cooreman W. et al: Bromelain Pharmacological Enzymes-Properties and assay method, (pg. 07-12; Ruyssen R. and Lauwers A. Story-Scientia Scientific Publishing Co. (1978)
12. Harrach T.; Eckert K.; Schulze-Forster K.; Nuck R.; Grunow D.; and Maurer H. Rainer: Isolation and partial characterization of basic proteinases from stem Bromelain. Journal of Protein Chemistry. Vol. 17: pg. 351-361 (1998).
13. Napper and Bennet, et al: Further purification and characterization of multiple forms of bromelainases derived cysteine proteinases Ananain and Comosain. (Biochemico. Journal. 301: pg. 727-735 (1994).
14. Lee K.; and Albee K. et al: Complete amino acid sequence of ananain and comparison with Bromelain and other plant cysteine proteinases (Biochemico. Journal 327: pg. 199-202 (1997).
15. Picker, I J, J, de los Toyos, M J Telen, B F Haynes, et al; In the Monoclonal antibodies antigens recognize the Hermes class of lymphocyte homing receptors. Journal of Immunology, 142, 2046 (1989).
16. Gallatin, W M, E. A. Wayner, P A Hoffman. et al: Structure homology between lymphocyte receptor for high endothelium and class III extracellular matrix. Proc. National Acad. Sct. USA 86, 4654 (1989).
17. Haynes B F, M J, Telen, L P, Hale et al; CD 44: a molecule involved in leucocyte adherence and T-cell activation.; Immunology Today 10: 423 (1989).
18. Denning S M, P T. Le, K H Singer and B F Haynes et al: Antibodies against the CD44 Lymphocyte homing receptor molecule augment human peripheral blood T-cell activation. Journal of Immunology, J. 44, 7 (1990).
19. Huet S, H. Groux B. Caillou, H. Valenton, et al; CD44 contributes to T cell activation. Journal of Immunology, J 43, 789 (1989).
20. Taussig, S. J.; Batkin, S.; Szekerezes, J.; et al.; Bromelain: the enzyme complex of pineapple (Ananas comosus) and its clinical application. An update, Journal of Ethnopharmacology, 22, pg. 191-203 (1988).
21. Kleef, R.; Delohery, T. M.; and Bovbjerg, D. J. et al; Selective modulation of cell adhesion molecules on lymphocytes by Bromelain protease-5. Pathobiology, 64, pg.339-346 (1996).
22. Maurer H, R; Hozumi, M.; Honma, Y.; and Okabe-Kado, J.; Bromelain induces the differentiation of leukemic cells in vitro: An explanation for its cytostatic effect? Planta Medica, 54, pg. 377-381 (1988).
23. Heinckie, R M.; van der Wal L.; and Yokoyama, M.; Effect of bromelain (ananase) on human platelet aggregation, Experientia 28: pg. 844-845 (1971).
24. Matsumoto, G.; Nghiem, M P.; Nozaki, N.; Schmits, R.; and Penninger, J M., Cooperation between CD44 and LFA-1/CD11a adhesion receptors in lymphokine-Activated killer cell cytotoxicity, Journal of Immunology,160, pg. 5781-589 (1998).
25. Mantovani, A.; Bottazzi, B.; Colotta, F.; Sozzani, S.; and Ruco, L.,: The origin and function of tumor-associated macrophages. Immunology Today, 13, pg. 265-270 (1992).
26. Eckert, Klaus; Grabowska, Edyta; Strange, Rainer; Schneider, Ulrike; Maurer, H. Raine; et al.: Effects of oral bromelain administration on the impaired Immunocytoxicity of mononuclear cells from mammary tumor patients, Oncology Report, 6: pg. 1191-1199 (1999).
27. Harrach T.; Eckert K.; Schulze-Forster K.; Nuck R.; Grunow D.; and Maurer H. Rainer: Isolation and partial characterization of basic proteinases from stem bromelain. Journal of Protein Chemistry 14: pg. 41-52, (1995).
28. Harrach T. ; Eckert K.; Schulze-Forster K.; Nuck R.; Grunow D.; and Maurer H. Rainer; Machleidt I.; and Machleidt M.; Isolation and characterization of two forms of an acidic bromelain stem proteinase; Journal of Protein Chemistry 20; pg. 53-64, (1997)
29. Van de Winkel, J G J.; Van Ommen, R.; et al., Proteolysis induces increased binding affinity of the moncyte type II FcR for human IgG, Journal of Immunology 143: pg. 571-578 (1989) 30. Batkin, S.; Taussig, S. J.; Szekerezes, J.; Antimetastatic effect of bromelain with or without its proteolytic and anticoagulant activity, Journal Cancer Res. Clinical Oncology.114, pg. 507-508 (1988).
31. Hale, I.; and Haynes, B F, et al; Bromelain treatment of human T-cells removes CD44, CD45RA, E2/M1C2, CD6, CD7 and Leu 8/Lam1 surfaces molecules and markedly enhances CD2-mediated T cell activation,: Journal of Immunology.149, pg. 3809-3816 (1992).
32. Taussig,S J.; Batkin, S.; and Szekerezes, J.: Inhibition of tumor growth in vitro by bromelain, an extract of the pineapple plant (ananas comosus), Planta Medica 6, pg. 538-539 (1985).
33. Ota, S.; Muta, E.; Katahira, Y.; and Okamoto, Y.: Reinvestigation of fractionation and some properties of the proteolytically active components of stem and fruit bromelain, Journal of Biochemistry, 98, pg. 219-228 (1985).
34. Rowan, A. D.; Buttle, D. J.; and Barrett, A. J. et al: Ananain, A novel cysteine proteinase found in pineapple stem: Arch. Biochemistry Biophysics, 267, pg.262-270 (1988).
35. Rowan, A. D.; Buttle, D. J.; and Barrett, A. J. et al: The cysteine proteinases of the pineapple plant, Biochemistry Journal, 266, pg. 869-875 (1990)
36. Yasuda, Y.; Takahashi, N.; and Murachi, T. et al: The composition and structure of carbohydrate moiety of stem bromelain, Biochemistry, 9, pg. 25-32 (1970).
37. Ishihara, H.; Takahashi, N.; Oguri, S; and Tejima, S, et al: Complete structure of the carbohydrate moiety of stem bromelain, Journal of Biology and Chemistry, 254;10715-10719 (1979).
38. Grabowska, E.; Eckert, K; Fichiner, I.; Schultze-Forster,K.; and Maurer, H. R., et al: Bromelain proteases suppress growth, invasion and lung metastasis of B16F10 mouse melanoma cells. International Journal of Oncology; 11, pg. 243-248 (1997).
39. Harrach, T.; Eckert, K.; Maurer, H R. et al: Isolation and characterization of two forms of an acidic bromelain proteinase; Journal of Protein Chemistry,17, pg. 351-361 (1998).
40. Garbin, F.; Harrach, T.; Eckert, K.; and Maurer H R.,et al: Bromelain proteinase F9 augments human lymphocyte-mediated growth inhibition of various tumor cells in vitro, International Journal of Oncology 5: pg. 197-203 (1994).
41. Harrach, T.; Gebauer F.; Eckert, K; Kunze, R.; and Maurer H. R, et al; Bromelain proteinases modulate the CD4 expression on human Molt 4/8 leukemia and SK-Mel28 melanoma cells in vitro. International Journal of Oncology, 5: pg. 485-488 (1994).
42. Eckert, K.; Grunberg, E.; Garbin, F.; and Maurer H R. et al: Preclinical studies with prothymosin a-1 on mononuclear cells from tumor patients, International Journal of Immuno pharmacology, 19: pg. 493-500 (1997).
43. Garbin, F.; T. Harrach, Eckert, K.; Buttner, P.; Garbe C.; Maurer H R, et al: Bromelain Proteinaes F9 augments human lymphocyte-mediated growth inhibition of various tumor cells in vitro.; International Journal of Oncology, 5, pg. 197-203. (1994).
44. Garbin F. T.; Harrach,; Klaus Eckert,; H. Rainer Maurer, et al; Prothymosin a-1 augments deficient antitumor activity of monocyte from melanoma patients in vitro; Anticancer Research 14, pg. 2405-2412 (1994).
45. Desser, L.; Rehberger, A.; Kokron, E.; and Paukovits, W. et al: Cytokine synthesis in human peripheral blood mononuclear cells after oral administration of polyenzyme preparation, Oncology 50, pg. 403-407 (1993).
46. Desser, L.; Rehberger, A.; and Paukovits, W. et al.: Proteolytic enzymes and amylase inducecytokine production in human peripheral blood mononuclear cells in vitro, Cancer Biotherapy 9: pg. 253-263 (1993).
47. Scambia, G.; and Ranelletti, F. O.; et al.: Quercetin inhibits the growth of multidrug-resistant estrogen receptor-negative MCF-7 human breast cancer cell line expressing type II estrogen-bnding sites: Cancer Chemotherapy, Pharmacology: 28, pg. 255-258 (1991).
48. Scambia, G.; and Ranelletti. F. O.; et al.,; Type II estrogen binding sites in a lymphoblastoid cell line and growth-inhibitory effect of estrogen, anti-estrogen and bioflavonoids: International Journal of Cancer: 46: pg. 1112-1116 (1990).
49. Ranelletti, F. O.; and Ricci, R.; et al.,; Growth Inhibitory effect of quercetin and presence of Type-II estrogen-binding sites in human colon cancer cell lines and primary colorectal tumors; International Journal of Cancer: 50, pg. 486-492 (1992).
50. Peterson, G.; and Barnes, S.: Genistein Inhibition of the growth of human breast cancer cells: Independence from estrogen receptors and multi-drug resistance gene: Biochemistry, Biophysics Research Commun.: 179, pg. 661-667 (1991).
51. Barnes, S.; et al.,; Effect of genistein on in vitro and in vivo models of cancer: Journal of Nutrition: 125, pg. 777S-783S (1995).
52. Guthrie, N.; and Moffatt, M.; et al.; Inhibition of proliferation of Human breast cancer cells by naringenin, a flavonoid in grapefruit: National Forum Breast Cancer, Montreal. Pg. 119 (1993).
53. Kandaswami, C.; and Perkins, E.; et al.: Anti-proliferative effects of citrus flavonoids on human squamous cell carcinoma in vitro: Cancer Lett.: 56, pg. 147-152 (1991).
54. Manach, C.; et al.,; Bioavailability, metabolism, and physiological impact of 4-oxo-flavonoids: Nutrition Research 16: pg. 517-544 (1996).
55. Harrach, Tibor,; and Eckert, Klaus; et al.,: Isolation and partial characterization of basic proteinases from stem bromelain: Journal of protein chemistry, Vol. 14, No.1, pg. 41-52 (1995).
56. Verma, A K.; Johnson, J A.; et al.: Inhibition 7,12-Dimethylbenza-anthracene and N-Nitrosomethylurea-induced of mammary cancer by dietary flavonoid Quercetin: Cancer Research: 48, pg. 5754-5758 (1988).
57. Castillo, M H.; and Perkins, E.; et al.: The effect of Bio-flavonoid-Quercetin on squamous cell carcinoma of head and neck origin, American Journal of Surgery: 158, pg. 351-355 (1989).
58. Singhal, R. L. ; and Yeh, Y. A.; et al., Qucercetin down-regulates signal transduction in human breast cancer cells: Biochemistry, Biophysics Research Commun., 208, pg. 425-431. (1995).
59. Yoshioka, S.; Izutsa, K.; Takeda, Y.; et al.,: Inactivation kinetic enzyme pharmaceuticals in aqueous solution; Pharmaceutical Research, 4: pg. 480-485 (1991)
60. Gunthert U.; Hofman M.; RUDY w.; Reber S.; et al; A new variant of glycoprotein CD44 Confers metastatic potential to rat carcinoma cells. Cell 65: pg. 13-24 (1991).
61. Birch M.; Miychell S.; and Hart I R.: Isolation and characterization of human melanoma cell variants expressing high and low levels of CD 44. Cancer Research 59:pg. 6660-6667 (1991).
62. Hofman M.; Rudy W.; Gunthert U.; et al; A link between rats and metastatic behavior of tumor cells: ras induces CD44 promoter activity and leads to low level expression of metastatic specific variants of CD44 in CREF cells. Cancer Research 53: pg. 1516-1521 (1993).
63. Maurer, H., and Eckert, K., Szekerezes, J., et al: Bromelain in the complementary tumor therapy; Journal of Oncology, 31: 66-73, 1989.
64. Kleef, R, Delohery, T. M. and Bovbjerg, D. J. et al; Selective modulation of cell adhesion molecules on lymphocytes by bromelain protease-5. Pathobiology, 64, pg. 339-346 (1996)
65. Maurer H. R.; Hozumi, M.; Honma, Y.; and Okabe-Kado, J, et al; Bromelain induces the differentiation of leukemic cells in vitro : an explanation for its cytostatic effect. Planta Medica, pp. 377-381 (1988).
66. Liao, Benedict; Liao, Alex; Liao, Austin; Liao, Judy; Liao, Burton; Liao, Justin; Liao, Edward; Liao, Susan; Liao, Joan; Liao, Ludwig, et al; Bioflavonoids derived from Fructus Crateagus served as anti-cancer and anti-lipidemic agents. (USPTO, Patent pending Publication, June, 2004)
67. Zhao, J., Wang, J., et al; anti-tumor promoting activity of a polyphenolic fraction isolated from grape seeds and identification of procyanidinB-5-3′-gallate as the most effective anti-oxidant constituent; (Carcinogenesis, Sept.; 20, (9) ; pg. 1737-1745, 1999)
68. Liao, Benedict; Liao, Justin; Liao, Edward,; Liao, Ludwig,; Liao, Susan, et al.; reported plant extracts derived from Ananas Comosus as an anti-cancer agents in animal experiments in breast and colonic carcinomas. (Internal communication, non-publication materials, 1984)
69. Filipova, Y., et al; In L-pyroglutamyl-L-Phenyl-Alanayl-L-Leucin-P-Nitroaniline, A chromogenic substrate for thio-proteinase assay.; Anal. Biochem. 143: pg. 293-297 (1984).
70. Rollwagen, et al.; Induces the secretion of Interlukin-IB, II-6, II -8, and tumor necrotizing factor (TNF) : Immunology Today :17; pg. 548-550 (1996).
71. Renzini et al.; Bromelain increases permeability of antibiotic drugs; Drug Research: 2; pg. 410-412 (1972).
72. Tinozzi, and Venegoni, et al.; Bromelain in the tissue permeability of antibiotic drug; Drug Exp. Clinical Research,; 1: pg. 39-44 (1978).
73. Netti, C., Bandi, G., et al.; Bromelain and its pharmacological effect on edema,: Pharmaco Ed. Pr. 8, 27: pg. 453-466 (1972).
74. Uhlig and Seifert, et al.; Bromelain and blood level of fibrinogen, and its fibrinolytic effects. Fortschritte Der Medizin, 15: pg. 554-556 (1981).
75. Smyth R., and Brennan, R., et al.; Plasmin activation and its relation with bromelain.; Arch Int. Pharmaco Dyn. 136: pg. 230-236 (1962).
76. Pirotta, et al,; Prolong the prothrombin and partial thromboplastin time in relative high doses of bromelain administration, Drug Exp. Clin. Research. 4: pg. 1-20, 1978. Gaeta
77. Livio, and De Gaetano et al,; Bromelain and its platelet aggregation effect; Drug Exp. Clin. Research, 1: pg. 49-53 (1978).
78. Morita, A. and Uchida, D. et al,; Inhibition of platelet aggregation in vitro with bromelain Treatment; Arch. Int. Pharmaco Dyn. 239: pg. 340-350 (1979).
79. Metzig, C., and Eckert, K., et al.; Bromelain prevents adhesion of platelet to endothelial cells of blood vessel; In. Vivo.: 13, pg. 7-12 (1999).
80. Seltzer, A. et al.: Bromelain reduces blood level of prostaglandine E-2, and thromboxane-A-2 in exudates during acute inflammation.: EENT. Monthly: 43; pg. 54-57 (1964).
81. Cantrell et al.; Microsomal study of Bromelain and its intracellular signal transduction namely, T-cell receptor (TCRs) DC2/CD3 signaling and Interleukin-II (IL-2) production, which are activated by Major Histocompatibility Complex (MHC) expressed on antigen presenting cells (APC).; Ann. Review Immunology, 14; pg. 259-274 (1996).
82. Mynott, A. and Engwerda, C.; et al: T-cell receptor (TCRs)/CD3, CD2 activated by bromelain through the Mitogen Activated Protein (MAP) Kinase Pathway. U.S. Pat. No. 7, 833,963 (2010).
83. Mark H. Beers; and Robert Barlow, Editors, Hyperlipidemia; Clinical, Biochemical and Pharmacological aspects; The Merck manual of Diagnosis and Therapy, 17^{th} edition, Merck Research Laboratories publishing: pg. 200-212 (1999).
84. Ross, R. et al.; The Pathogenesis of atherosclerosis: a perspective for the 1990s, Nature; 362, pg. 801-809 (1993).
85.Stary, H. C. and Chandler A.B. et al.; A definition of advanced typed of atherosclerotic lesions and histological classification of atherosclerosis. A report from the Committee on Vascular lesions of the Council on Atherosclerosis, American Heart Association. Circulation, 92: pg. 1355-1374 (1995).
86. Lubert, Stryer. Author,; Textbook of Biochemistry, 3^{rd} edition, Chapter 23, Biosynthesis of lipids. W.H. Freeman and Co. Publishing: pg. 554-566 (1988).
87. Witiak, D. T. and Feller,D. R. et al.; Antilipidemic Drugs: Medicinal, Chemical and Biochemical Aspects; Elsevier, pg. 159-195 (1991).
88. Gerald K. McEvoy,: Editor, Antilipidemic agents: Clinical, Biochemical and Pharmacological aspects, Drug Information: American Society of Health System Pharmacists publishing: pg. 1430-1468 (1998) and pg. 1705-1750 (2001)
89. Revilla E, and Ryan J M. Et al.; Analysis of several phenolic compounds with potential antioxidant properties in grape extracts and wines by high-performance liquid chromatography-Photodiode array detection without sample preparation,: Journal of Chromatographia, 6,881(1-2); pg. 461-469 (2000)
90. E. Reinhard,; Tubingen Editor-in-Chief, Ammon, H. P. T. and Haendel, M. et al., Planta Medica, Toxicology and Pharmacology of Crataegus : Journal of Medical Plant Research: 43 (2) pg. 105-120,: 43(3) pg. 210-239: 43 (4) pg. 316-323 (1981)
91. D. A. Rakotoarison et al. Antioxidant activities of Polyphenolics extracts from flowers of Craetaegus Monogyna. Pharmazie; 52: pg. 60-64 (1997)
92. Bayler T. et al., Bioflavonoids and its anti-inflammatory effects; Phytochemistry, 28, pg. 2373-2378 (1989)
93. Kaul, T. N. and Elliot Middleton et al.; Antiviral effect of Citrus Flavonoids on human viruses, Journal of Medical Virology: 15; pg. 71-79 (1985)
94. Saija, A. and Scalese, M. et al.: Flavonoids, Quercetin, Hesperetin, Naringenin, and Rutin, as Anti-oxidant agents: Free Radical Biology and Medicine: vol. 19, no. 4, pg. 481-486 (1995).
95. Felicia V. So, and Najla Guthrie et al., Inhibition of human breast cancer cell proliferation and delay of mammary tumor-genesis by Flavonoids and citrus juices: Nutrition and Cancer, 26 (2), pg. 167-181 (1996).
96. Bok, S. H. and Jeong, T. S. et al.: Flavonoids derived from citrus peel as collagen induced platelet aggregation inhibitor: U.S. laid open Pat. No. 6,221,357.
97. Nair, M. G. and Wang, H. B. et al.: Method of inhibiting cyclooxygenase and inflammation using cyanidins: U.S. laid open Pat. No. 10,002,407.
98. Shanthi S. et al.: Hypolipidemic activity of tincture of Crataegus in rats: Indian Journal of Biochemistry and Biophysics; 31(2), pg. 143-146 (1994).
99. Rajendran S. and Deepalakshmi P. D. et al.: Effect of tincture of Crataegus on the LDL-Receptor Activity of hepatic plasma membrane of rats fed an atherogenic diet. Atherosclerosis: 123: pg. 235-241 (1996)
100. Wang S. L. and Li, Y. D.; et al.: Inhibition of 3-HMG CoA Reductase activity in hepatic and intestinal mucosal cells in guinea pig fed with concentrated aqueous extract of Crataegus Pinnatifida; Journal of Traditional Chinese Medicine: 7;(8): pg. 483-484 (1987)
101. Zhao J.; Wang J.; Chen Y.; and Agarwa R.: Antitumor promoting activity of a polyphenolic fraction isolated from grape seeds in the mouse skin two-stage initiation-promotion protocol and identification of procyanidin B5-3′-gallate as the most effective antioxidant constituent. Carcinogenesis, 1999 Sept; 20(9); pg. 1737-1745.
102. Guan Y.; Zhao S. et al.; Blood lipid tablets in the treatment of Hyperlipidemia: Journal of Traditional Chinese Medicine: 15 (3), pg. 178-179 (1995).
103. Scambia, G. and Ranelletti, F. O. et al., Quercetin inhibits the growth of a Multidrug-Resistant Estrogen Receptor-Negative MCF-7 Human Breast Cancer cell line Expressing Type II Estrogen-Binding sites; Cancer Chemotherapy, Pharmacology: 28, pg. 255-258 (1991)
104. Scambia, G.; and Ranelletti, F. O.; et al., Type II Estrogen binding sites in a Lymphoblastoid cell line and Growth-inhibitory effect of Estrogen, Anti-Estrogen and Bioflavonoids; International Journal of Cancer:46: pg. 1112-1116 (1990).
105. Singhal, R. L. and Yeh, Y. A.; et al.: Quercetin Down-Regulates Signal Transduction in Human Breast Cancer cells: Biochemistry, Biophysicis Research Commun.: 208, pg. 425-431 (1995)
106. Castillo, M. H.; and Perkins, E. et al.; The effect of the Bioflavonoid-Quercetin on Squamous cell Carcinoma of Head and Neck origin, American Journal of Surgery: 158, pg. 351-355 (1989).
107. Ranelletti, F. O. and Ricci, R. et al.: Growth Inhibitory effect of Quercetin and presence of Type-II Estrogen-binding sites in human colon cancer cell lines and primary colorectal tumors; International Journal of Cancer: 50, pg. 486-492 (1992).
108. Verma, A K.; Johnson, J A.; et al.: Inhibition of 7,12-Dimethylbenza-anthracene and N-Nitrosomethylurea-induced rat Mammary Cancer by Dietary Flavonoid Quercetin; Cancer Research: 48, pg. 5754-5758 (1988).
109. Ammon, H. P. T. and Haendel, M. et al.: Crataegus Toxicology and Pharmacology, Planta Medica: 43(2): pp. 105-120, 43(3): pp. 210-239, 43(4): pg. 316-323 (1981).
110. Schussler, M, and Holzl, J.; et al.: Increasing Cardiac Perfusion with Quercetin, Rutin, and Vitexin in guinea pig heart: Arzneimittelforschung, 45(8): pg. 842-845 (1995).
111. Manach, C. et al.: Bioavailability, Metabolism, and Physiological impact of 4-oxo-Flavonoids: Nutrition Research 16: pg. 517-544 (1996).
112. Hertog, M. G. L. et al., Dietary Anti-oxidant Flavonoids and risk of coronary heart disease: Lancet: 342: pg. 1007-1011 (1993).
113. Peterson, G, and Barnes, S. et al.: Genistein Inhibition of the growth of Human Breast cancer cells: Independence from Estrogen receptors and the Multi-drug resistance gene: Biochemistry, Biophysics Research Commun.: 179, pg. 661-667 (1991)
114. Barnes, S. et al., Effect of Genistein on In Vitro and In Vivo Models of Cancer: Journal of Nutrition: 125, pg. 777S-783S (1995).
115. Guthrie, N. and Moffatt, M. et al., Inhibition of proliferation of Human Breast Cancer cells By Naringenin, a Flavonoid in Grapefruit: National Forum Breast Cancer, Montreal. P 119 (1993).
116. Kandaswami, C. and Perkins, E. et al.: Antiproliferative effects of Citrus Flavonoids on Human Squamous Cell Carcinoma in Vitro: Cancer Lett.: 56, pg. 147-152 (1991).
117. Hulcher, F. H. and Oleson W. H. et al.: Simplified Spectrophotometric assay for microsomal 3-hydroxy-3-methyl-glutaryl-Co enzyme A reductase by measurement of coenzyme A. Journal of Lipid Research; 14, pg. 625-641(1973).
118. Fogt F. and Nanji A. et al.: Alterations in nuclear ploidy and cell phase distribution of rat liver cells in experimental alcoholic liver disease: relationship to antioxidant enzyme gene expression. Toxicology and applied Pharmacology; 136 (1) pg. 87-93(1996).
119. Keegan A. and Martini R. et al.: Ethanol-related liver injury in the rat: a model of steatosis, inflammation and pericentral fibrosis, Journal of Hepatology; 23 (5), pg. 591-600 (1995).
120. Fleming, Thomas. Chief editor, Crataegus laevigata; Physician Desk Reference for Herbal Medicine, 2nd edition, Medical Economics Co. publishing; pg. 271-275(2000).
121. Lee-Huang S. et al; Proc. Natl. Sci. USA. 81, pp 2708-2912 (1984)
122. Sanger, F., Nicken, S. & Coulson, A. R.; Proc. Natl. Sci. USA, 74, pp 5463-5467(1977).
123. Okayama, H. & Berg, P. Mol. Cell. Biol. 2, pp 161-170(1982).
124. Gasser, C. S., Simonsen, C., Schilling, J. W. & Schmike, R. T.; Proc.Natl.Sci. USA 79, pp 6522-6526 (1982).
125. Woo, S. L. C. Methods Enzymol.; 68, pp 389-395 (1979)
126. Maxam A. M. & Gilgert, W.; Mothods Enzymol. 65; pp 499-560 (1980).
127. Lin, F. K., Lai, P. H., Smalling, R., Egrie, J., Browne, J., Lin, C. H., Wang, F. F. & Goldwasser, E.; Exp. Hematol.; 12, pp 357 (abstr. 1984).
128. Miyake, T. Kung, C. K. H. & Goldwasser, E.; Journal Biol. Chem. 253, pp 5558-5564 (1977). 129. Lawn, R. M., Fritsch, E. F., Parker, R. C., Blake, G. & Maniatis, T. Cell. 15; pp 1157-1174 (1978).

### BACKGROUND OF THE INVENTION

Cloning and expression of the gene of the human erythroblast cell has been initiated by several institutes in early 1970, Dr. Lee-Huang from University of New York in 1984 successfully cloning the erythroprotein from human erythroblast cells (Proc. Natl. Sci. USA. 81, 2708-2712). Fu-K, Lin,; Sidney Suggs,: Eugene Goldwasser, et al also successfully cloning the erythroprotein from human erythroblast cells and put into commercialized production.(Proc. Natl. Sci. USA. 82; 7580-7584). Present inventors have been cloning and expression in Comosains minigene and put it to clinical usage.

Comosain represents 80% of Bromelainases component,{(Dr. Henry Mauror, Harrach T. Eckert K, Schulze-Forster K., Nuck R. et al ; Isolation and partial characterization of basic proteinases from stem bromelain, Journal of protein Chemistry Volume 14, pp 41-52 (1995) : Volume 17; pp 351-361 (1998),; Volume 20; pp 53-64 (1997)). The other forms of bromelain proteinases also include Ananase (Bromelain F-9a)(represent 10%), Bromelain-F4, Bromelain-F5, Bromelain F-9b (Comosain), and Bromelain F2, F3, F-6, F-6, F-7 and F-8(H. R. Maurer, Bromelain: Biochemistry, Pharmacology and Medical use; CMLS, Cell. Mol. Life Sci. 58; pp 1234-1245 (2001). (ok)

The Comosains gene has been isolated from a genomic phase library by using mixed 20 mer and 10 mer oligonucleotide probes. The entire coding region of the gene is contained in a 4.8-Kilobase Q9S8M1-Blast V to Blast-G fragment. The gene contains four intervening sequences (3026 base pairs) and four exons (1816 base pairs). It encodes 20 amino acid signal peptides with calculated Molecular mass of 23,509 to 23,569. The Comosain protein gene, when introduced into New Zealand white rabbit ovarian cells produces Comosain that is biologically active in vitro and in vivo.

Administered of oral bromelain in cancer treatment in nonclinical trials has been reported as early in 1968 by Wolf M, & Ransberger k.¹, In vitro and animal studies have suggested of anti-metastatic effect for bromelain. Batkin &Taussig^{2,3} in 1988 reported that orally administered bromelain reduced the incidence of pulmonary metastasis in Lewis lung cancer cells in mice, In recent years, 1988 Batkin & Taussig⁴ suggested the antitumor mechanisms are due to fibrinolytic effect in Bromelain. Taussig & Batkin in 1988⁵ discovered that Bromelain has anti-platelet aggregation effects. Taussig and Batkin in 1985⁵ also discovered Inhibition the growth of tumor cells such as Lewis lung carcinoma, V-8 lymphoma, MC1-1 acites, KATO-gastric carcinoma cells. Maurer, & Hozumi, in 1994⁶ Discovered bromelian Induced Differentiation in leukemic cells. Hale, & Haynes in 1992⁷ and Cantrell et a in 1996 have been suggested that MMAPT(Major Mitogen Activating Protein Kinase) and TPK (Tyrosine Phosphorylation Kinase) inhibitors were activated by Bromelain. T-cell activation and cascade production of Interleukin II-B, 6, 8, and TNF-a (Tumor Necrotizing Factors) via CD-2, CD-3 surface antigen of WBC.

Garbin, Harrach, Eckert, & Maurer in 1994¹⁵ and Hale, & Haynes in1992⁷ also suggested that Bromelain will reduce surface antigens of CD-44, CD-44 v, CD-44s, CD45, & CD 47 in tumor cells of breast carcinoma.

From the experimental studies above, we conclude that activation of Bromelain proteinases in lymphocytes and T-cells have anti-metastatic effects both in vitro and in vivo.

According to the recent studies and reports, various types of cancer and neoplastic diseases have been amount the number one cause of deaths in America. The search for new medicines with non-toxic, low side effects which are compatible with most other drugs have been under way since Cohen in 1964, Renzini in 1972, and Tinozzi in 1978. Bomelain was first isolated from Ananas Comosus fruit in 1891 by Marcano. (Marcano. Bull. Pharma. 5, 77, (1891). Heinecke and Gortner discovered stem bromelain, as a new proteinase preparation from Ananas Comosus plant in 1957, by precipitating with acetone and with ammonium sulfate in 1961. Further purification of crude preparation was formulated by Gibian and Bratfish in 1960 and a patent right was granted to Pineapple Research Institute (U.S. Pat. No. 3,002,891) and to AG Schering Company (U.S. Pat. No. 2,950,227). Bayer T., reported the anti-inflammatory effects in flavonoids in 1989 (Phytochemistry, 28, pg. 2373-2378 (1989).

Utilization of plant and/or fruit flavonoids such as Quercetin, Rutin (Quercetin -3-Retinoside), Naringenin and Hesperetin, Genistein, in inhibiting tumor growth have been reported by Saija A., Scalese M. et al., (Free Radical Biology and Medicine vol. 19, no. 4 pg. 481-486 (1995)), Felicia, V. S.;, Najla Guthrie, et al. depicted evidence of inhibition of human breast cancer cell proliferation and delay of mammary tumor-genesis by flavonoids and citrus juices.(Nutrition and Cancer vol. 26, no. 2, pg. 167-181 (1996)).

In the year 2000, Revilla, E., and Ryan J. M., et al. analyzed of several phenolic compounds with potential antioxidant properties in grape extracts and wines by high-performance liquid chromatography-photodiode array detection without sample preparation. (Journal of Chromatographia; 6, 881 (1-2); pg.461-469 (2000)). Also in 1991 by Kandaswai, C. and Perkins E. et al reported that citrus flavonoids have anti-proliferative effects on human squamous cell carcinoma in vitro: (Cancer Lett.; 56, pg. 147-152 (1991). Guthrie, N., and Moffatt, M., et al claimed Naringenin, a flavonoid from grapefruit, has anti-proliferative effect in human breast cancer cell lines. (National Forum Breast Cancer, Montreal, pg. 119 (1993)).

Bioflavonoids are group of naturally occurring compounds, which have a common flavone nucleus composed of two benzene rings linked through a heterocyclic-pyrone ring. They are found plentifully in various plants, vegetables, fruits (such as; citrus fruits, grapes), food products (such as buckwheat and oatmeal) and dyes of natural origin. Bioflavonoids exhibit various biochemical and pharmacological activities including anti-oxidative, anti-inflammatory, anti-cancer, anti-viral, and anti-platelet aggregation. (D A. Rakotoarison et al. Antioxidant activities of polyphenolic extracts from flowers of Crataegus monogyna. Pharmazie; 52: pg. 60-64 (1997)), (Bayler, T.,et al., Phytochemistry 28, 2373-2378 (1989)), (Goda,Y.,et al., Chem. Pharm. Bull. 40, pg. 2455-2457(1992)), (T N. Kaul and Elliott Middleton et al., Antiviral effect of Citrus flavonoids on human viruses, Journal of medical virology :15; pg. 71-79(1985)), (A. Saija and M. Scalese et al., four flavonoids, Quercetin, hesperetin, naringenin, and rutin, as anti-oxidant agents; Free radical biology and medicine, vol 19, no. 4, pg. 481-486(1995)),(Felicia V. So. and Najla Guthrie et al., Inhibition of human breast cancer cell proliferation and delay of mammary tumor-genesis by flavonoids and citrus juices, Nutrition and cancer vol. 26, no. 2, pg. 167-181(1996)), (S H. Bok and T S. Jeong et al., Flavonoids derived from citrus peel as collagen induced platelet aggregation inhibitor, U.S. Laid open Pat. No. 6,221,357), (M G. Nair and H B. Wang et al., method of inhibiting cyclooxygenase and inflammation using cyanidin, U.S. Laid-open Pat. No. 10,002,407)

Quercetin (3,5,7,3′,4′ penta-hydroxy flavone) also has anti-cancer activities against breast cancer, colon cancer cells, lymphoblastoid cell lines and squamous cancer cell lines and anti-viral activities against herpes simplex type I, polio virus type I, para-influenza type-3, and respiratory syncytial virus. (Scambia, G. and Ranelleti, F. O. et al., Cancer Chemotherapy, Pharmacology: 28, pg. 255-258 (1991)), (Singhal, R. L. and Yeh, Y. A. et al., Biochem. Biophys. Research Commun. 208, pg. 425-458 (1995)), (Ranelleti, F. O. and Ricci, R. et al., International J. of Cancer: 50; pg. 486-492 (1992)), (Scambia, G. and Ranelleti, F. O. et al., International J. of Cancer: 46; pg. 1112-1116 (1990)), (Castillo, M H. and Perkins, E. et al., American J. Surgery 158: pg. 351-355 (1989)), (Verma, A. K. and Johnson, J. A. et al., Cancer Research 48: pg. 5754-5758 (1988)), (Kaul, T. N. and Middleton, E. J. et al., Journal of Medical Virology 15: pg. 71-79 (1985)), (Japanese Laid-open Patent No. 4-234320)

Rutin, a glycosylated quercetin, (Quercitin-3-rutinoside) is decomposed in the intestine by microorganisms and absorbed in the intestine, Vitexin (Apigenin, Orientoside, or Flavone, 8-D-glucosyl-4′,5,7-tri-hydroxy-), and Kaempherol (3,5,7,4′ tetra-hydroxy-flavone) also have anti-hypertensive properties and increase coronary and cardiac perfusion. (Ammon, H. P. T., and Haendel, M. et al., Crataegus toxicology and Pharmacology, Planta medica: 43(2): pg. 105-120, 43(3): pg. 316-322, and 43(4): pg. 210-239 (1981)), (Schussler, M. and Holzl, J. increasing cardiac perfusion with quercetin, rutin and vitexin in guinea pig heart, Arzneimittelforschung, 45(8): pg. 842-845 (1995)), (Manach, C. et al., bioavailability, metabolism, and physiological impact of 4-oxo-flavonoids. Nutrition research 16: pg. 517-544(1996)). Furthermore, Hertog et al. reported that high intake of rutin and quercetin in the food product may reduce coronary heart disease related death rate of elderly patients (M. G. L. Hertog et al., dietary antioxidant flavonoids and risk of coronary heart disease, Lancet: 342: pg. 1007-1011 (1993)).

The present inventors have discovered that bioflavonoids derived from hawthorn berry, and citrus fruits such as rutin, quercetin, kaempherol, vitexin, hesperetin, naringenin, genistenin are effective in treating and/or preventing various types of cancer.

The study of Rakotoarison, D A. et al showed anti-oxidant activity of polyphenolic extracts from flowers of Crataegus Monogyna (Pharmazie: 52: pg. 60-64 (1997)). Kaul, T N. and Elliott Middleton et al also reported the anti-viral effect of Citrus flavonoids on human viruses. (Journal of Medical Virology: 15; pg. 71-79 (1985)). It has also been reported that pant bio-flavonoids exhibit various biochemical and pharmacological activities including anti-oxidant, anti-inflammatory, anti-cancer, anti-viral and anti-platelet aggregation.

Nair, M G; and Wang, H B. et al reported that a method of inhibiting cyclooxygenase and inflammation using cyaniding, (U.S. Pat. No. 10,002,407)), Ranelleti, F. O., and Ricci R. et al, reported growth inhibitory effect of Quercetin and presence of type II estrogen binding sites in human colon cancer cell lines and primary colo-rectal tumors; (International Journal of Cancer: 50; pg. 486-492 (1992)), Scambia, G. and Ranelleti, F. O. et al ; reported that Quercetin inhibits the growth of a multi-drug-resistant estrogen receptor-negative MCF-7 human breast cancer cell line expressing type-II estrogen-binding sites: (Cancer Chemotherapy, Pharmacology: 28, pg. 255-7.58 (1991)) also reported in Type II estrogen binding sites in a lymphoblast cell line and growth inhibiting effect of estrogen, anti-estrogen and bioflavonoids; (International Journal of Cancer: 46; pg. 1112-1116 (1990)), Castillo, M. H. and Perkins, E., et al also reported the effect of the bioflavonoid-Quercetin on squamous cell carcinoma of head and neck origin; (American Journal of Surgery: 158, pg. 351-355 (1989)).

Zhao, J., Wang, J., et al published a study showing anti-tumor promoting activity of a polyphenolic fraction isolated from grape seeds and identification of Procyanidin B 5-3′-gallate as the most effective antioxidant constituent; (Carcinogenesis, Sept.; 20 (9); pg. 1737-1745 (1999)).

In 1995 Harrach T., Eckert K., Schulze-Forster K. and Maurer H. Rainer et al reported isolation and partial characterization of basic proteinases from stem bromelain. (Journal Protein Chemistry 14: pg. 41-52, 1995), and Again in 1997 they reported isolation and characterization of two forms of an acidic bromelain stem proteinase.(Journal Protein Chemistry 20: pg. 53-64, 1997).

In 1985 Taussig, and Batkin et al suggested that the enzyme complex from Ananas Comosus produced inhibition of tumor growth in vitro (Planta Med. 6: 538-539, 1985). and outlined its clinical application,(Journal Ethnopharmacology: 22, pg. 191-203,1988) The same enzyme complex also produces platelet anti-aggregation, and fibrinolytic activity, (reported by Heinecke and Yokoyama in 1957, Heinecke in 1972, Marz, in 1982,). In 1979 Klaue Amen, and Roman et al reported use of bromelain as chemical debridement agent in 3^{rd} degree burn patients.(European Surgical research, 1979, pg. 355-359). In same year of 1988 the same team also discovered that Bromelain has an anti-metastatic effect with or without its proteolytic and anti-coagulant activity, (Journal of Cancer Research Clinical Oncology, 114: pg. 507-508, 1988).

The present inventors have also discovered that Glco-polypeptides, the extract of Bromelain proteins which derived from the fruit and stem of Ananas Comosus. (Comosain, Bromelain, Ananase, Inflamen, Extranase, Traumanase) which are effective in treating and/or preventing various types of cancer and neoplastic diseases including breast, colon, lung, ovarian, cervical, and uterine cancers, and so forth. the method comprises administered an effective amount of Comosain, Bromelains, Ananase, Pepsin, Trypsin., Naringenin, Hesperetin, Genistin and/or a mixture thereof. By the following attributes:
(a) Anti-inflammatory properties
(b) Anti-platelet aggregation (Mynott et al in 1998, suggested that it changes the tumor surface antigen thus preventing tumor cells from attacking the normal tissues),
(c) Fibrinolytic properties,

(d) Anti-tumor genesis, this action was probably due to release of tumor necrotizing factors (TNFs) in T-cells of WBC. Taussig et al in 1985 and Taussig, and Batkin et al in 1988 both indicated that Bromelain and Comosain extract can be used in inhibiting tumor growth, the T-cells, peripheral blood mononuclear lymphocytes (PBMN) with influence of bromelain, they produce and outpour of TCRS/CD2, TCRS/CD3, Interlukin IIB, II-6, II-8, and TNFa. and attack the tumor surface antigens of CD44, CD44-v, CD44-s, CD45, CD47. These Action mechanisms are through two major pathways:
(d.-1) Major Mitogen Activating Protein Kinases (MMAP) inhibitors.
(d-.2) With the engagement of Bromelain (Antigen Presenting Polypeptide) (APP) (presented by Major Histocompatibility Complex (MHC) expressed on the Antigen Presenting Cells (APC)(Cantrell 1996)
(d-3) Tyrosine Phosphoration Kinase inhibitors.
(e) Anti-dedifferentiation both in vitro and in vivo in cancer cell lines, in animal, and human experiments.

The present invention and discovery relates to the methods and compositions for treating and/or preventing various types of cancer and neoplastic disorders in mammals, which comprises administration thereto of an effective amount of Glycopolypeptides (such as Comosain, Bromelainases, Ananase, Pepsin, Trypsin) and Bioflavonoids such as Quercetin; Rutin (Quercetin-3-Rutinoside), Hesperetin, Genistein, Naringenin and/or a mixture thereof.

### BRIEF DESCRIPTION OF THE DRAWINGS

The objects of the present invention will become increasingly apparent by reference to the following descriptions and drawings.

Assembly of Expression Vector for the Comosain Gene and Preparation of Genetically Modified Organism (GMO)

The oligo-deoxyribonucleotide probe synthesis was used, the phosphoramidite method was used for oligonucleotide synthesis. Each probe mixture contained a pool of 20-oligonucleotides sequences were:

Probe mixture: CoM-V sequence equivalent to: valine-proline-glutamine-serine-isoleucine,
aspartic acid-tryptophan-arginine-asparagine-tyrosine-glycine-alanine-valine-threonine-
serine-valine-lysine-asparagine-glutamine-glycine.
Probe mixture: CoM-V =
Val-Pro-Glu-Ser-Iso-Asp-Trp-Arg-Asn-Tyr-Gly-Ala-Val-Thr-Ser-Val-Lys-Asn-Glu-Gly
3′ CAA, GGA, GTT, --------------------------------------------------------, TTG, GTT, TT5′
V D G V
P W A K
Q R V N
S N T Q
I Y S G
V = Valine, P = Proline, Q = Glutamine, S = Serine, I = Isoleucine.
D = Aspartic acid, W = Tryptophan, R = Arginine, N = Asparagine, Y = Tyrosine.
G = Glycine, A = Alanine, V = Valine, T = Threonine, S = Serine.
V = Valine, K = Lysine, N = Asparagine, Q = Glutamine, G = Glycine.

The probe mixtures were labeled at the 5′ end with [r³²p] ATP, 7500-8000 Ci/mmol (ICN) (1 Ci=37 GBq), by using T4 polynucleotide kinase.

Hybridization procedures: phage plaques were ampilified according to the procedures of Woo, except that GeneScreen Plus filters and NZYAM plates [NACL, 5 g; MgCl₂-6H20, 2 g; NZ-Amine A, 10 g; yeast extract, 5 g; Casamino acids, 2 g; maltose, 2 g; and agar, 15 g (per liter) were used, Phage particles were disrupted and the DNAₛ were fixed on filters (50,000 plaques per 8.4×8.4 cm filter). The air dried filters were baked at 80° C. for 1 hour and then subjected to proteinase K digestion [50 ug of proteinase k per ml of buffer solution containing 0.1M Tris-HCl (PH 8.0), 0.15 M NaCl, 10 mM EDTA, and 0.2% NaDodSo4 for 30 min at 55° C. Prehybridization with a 1 M NaCl/1% NaDodSo4 solution was carried out again at 55° C. for 4 hours or longer

The hybridition buffer contained 0.025 pmol/ml of each of the 20 probe sequences of 0.9M NaCl/5 mM EDTA/50 m M solution phosphate, PH 6.5/0.5% Na Dod So4/100 ug of yeast tRNA per ml. Hybridization was carried out at 48° C. for 20 hrs by using the ComV probe mixture. (that is 2° C. below the lowest calculated dissociation temperature (td) for members of The mixture. At the completion of hybridization, the filters were washed three times with 0.9 M NaCl/90 mM sodium citrate, pH 7.0/0.1% NaDodSo4 at room temperature at hybridization and 10 min per wash.

For direct expression of the genomic Comosain gene, 4.8 kilobase (kb) of BstTy/Se- and BamAs/GL fragment of Comosain, which contains the entire gene. After converting the BstTy/Se (tyrosine-serine 10 amino acids)site into Bst As/GL(asparagine-to-glycine 20 amino acids) site with a synthetic linker(pBR322 ori) the fragment was insert into the unique BamAs/GL site of the expression vector pDSVL, which contains a dihydrofolate reductase (DHFR) minigene. The resulting Plasmid DSVL-gPlCOS (gene Plant Comosain) was then used to transfect New Zealand white rabbit ovarian (NWRO) cells by the calcium phosphate microprecipitate method. The transformants were selected by the medium lacking hypoxanthine and thymidine. The culture medium used was Dulbecco's modified Eagle's medium supplemented with 10% fetal calf serum, penicillin, streptomycin, and glutamine.

Isolation of Comosain mRNA.: The 4.8 kilobase Bst ty/se-Bam As/GL restriction fragment from Comosain was inserted into shuttle vector, pSV4ST. The resulting chimeric plasmid pSV gPLComo was used to transfect Cos-1 cells by the calcium phosphate micoprecipitate method. After culture for 72 hrs, RNA was prepared from the transected cells by the guanidinium thiocyanate procedure of Chirgwin et al. and poly(A)+ mRNA was isolated by binding to oligo-cellulose.(Aviv & Leder).

cDNA cloning.: A Comosain cDNA bank was constructed according to a modification of the general procedures of Okayama and Berg by using the poly(A)+ mRNA described above (Mol. cell biology 2, 161-170, 1982).

DNA sequencing. : Restriction fragments were cloned into M 13 phage vectors by using Escherichia coli strains JM 103 and/or JM 109 as host. (Messing, J. of methods enzymology 1983) and were sequenced by the dideoxy method of Sanger et al. some regions were sequenced by kinase labeling or end-fill labeling of restriction fragments followed by chemical cleavage as described by Maxam and Gilbert. (J. of methods of enzymology 1980).

The final Comosain Recombinants were collected, extracted, washed with ethyl alcohol, and purified by AKAT Prime (GE Co.) and/or FPLC-cation-exchange chromatography, to produce of F4, F5, and F-9a, F9b (Comosain and Ananase) for future use.

Table I, Overview of the Bromelains (Comosain) that can be used in inhibition of these characterized cancer cells.

Six types of cancer cell lines were used in our present invention and discovery, which included breast, lung, colon, cervical, ovarian, and uterine cancers, and so forth, Most of our cancer cell lines were harvested directly from surgical specimens during surgery, and were prepared and described in detail under cell culture in Example -2.

Materials and Methods: Naringenin, Hesperetin, Genistein, Trypsin, Pepsin, & Bromelain (Comosain) proteinase molecules were purchased from Sigma-Aldrich Co. St. Louis, Mo.(for cell cultures) (Catalogue No#4882 & more), Complete Growth Medium (Catalogue #M4655), Tween-20, and Tween-80 solution (Catalogue No #P2287, #P 8192), Penicillin-streptomycin-Neomycin Stabilized Solution (Catalogue No#P 4083), Fetal calf serum (Catalogue No #4762). Cell culture wares were from Becton-Dickinson Co, Franklin Lakes, N.J., (Catalogue No #353503,), B-D™ Cell viability kit with liquid counting beads (Catalogue No #349486), B-D FACS Array™ Bio-analyzer (Catalogue No #340128). Pathological and microscopic images were taken by Amescope Trinocular Microscope (American Optic Co., Model No#T-490B-10M).

The complete growth medium (CGM): consists of Dulbecco's modified essential medium (Sigma-Aldrich Co., St. Louis, Mo.), supplemented with 10% heat inactivated fetal calf serum, 2% L- glutamine, penicillin(100 iu/ml), streptomycin (5 mg/ml), and neomycin (10 mg/ml) (Sigma-Aldrich Co. St. Louis, Mo.).The cells were maintained in a standard tissue culture incubator at 37° C. with atmosphere humidity of 90% air, and 10% CO 2. All cancer cell lines were initiated by seeding 5×(10)⁶ cells into 75 cmsq tissue culture flasks and base were coated with 0.75% agar in CGM. The cancer cell lines were used between 5 and 7 days of culture.

FIG.-IA, Growth inhibition of various types of tumor cell lines in vitro. Bromelain in medium in mg/ml in X-axis, Percentage % of cell growth VS control in Y-axis. Inhibition of tumor cell growth with increasing Bromelain concentration in all six cancer lines: breast, colon, lung, ovarian, cervical, and uterine cancers. The cells in all groups were incubated for 72 hours at 37C degree in 10% CO2 in air and were counted with Coulter counter. The graph represents the six individual experiments with 6 tumor cell cultures/each tumor line in each experiment.

FIG.-IB, The methods of analysis of surface antigens production of interleukin IB, IB6, II8, TNF-α, in TCRS/CD2, TCRS/CD3 in T-cells, and Mononuclear Cells. In WBC cell-lines culture fluid of 5×(10)⁶. At the concentration of 1 mg/ml of Bromelain (Comosain) installation to produce of interleukin IIB was 13,000 pgm/ml/(10⁶) WBC (increased by 400 folds), interleukin II6 was 26,000 pgm/ml/(10)⁶ WBC (increased by 650 folds), TNF-α was 1500 pgm/ml (10)⁶ WBC (increased by 42 folds).

FIG.-I-C, Depicts that of surface antigens of CD44-s, CD44-v, modulation with two different mAbs clones, L-178, I-173. Breast cancer cells were incubated for 1 hr at 37 C with 10 ug/ml, 50 ug/m[, 75 ug/ml of Bromelain (Comosain) treatment.(Using radioimmuno monoclonic antibody tests). The CD44s become 35%, 10%, and 0% of Bromelain treated cells. The CD44v become 33%, 11%, and 0% of Bromelain treated cells

FIG.-ID, Inhibition of tumor cell growth of lung cancer by pepsin and trypsin in vitro., The cells in these two group were incubated for 72 hours at 37 C degree in 10% CO2 in air and were counted with Coulter counter. The graph represents the two experiments with Pepsin and Trypsin.

FIG. IE, Growth inhibition of breast cancer cell line by Qucercetin and Naringenin in vitro.

Rutin (C.sub.27.H.sub.30. O.sub.16, Molecular weight of 610.52, Glycosylated Quercetin, or Quercetin-3-rutinoside); Quercetin (C.sub.15. H.sub.10. O.sub.7, Molecular weight of 302.24, or 3,5,7,3′,4′ penta-hydroxy flavone); Kaempherol (C.sub.15. H.sub.10. O.sub.6, Molecular weight of 286.24, or quercetin-3-rhamnoside); Vitexin (Apigenin)(C.sub.21.H.sub.20.O.sub.10, Molecular weight of 432.38, or 8-D-glucosyl-4′,5,7 trihydroxy-flavone) (Merck Index 13rd Edition 2001) may be extracted from various plants, vegetables and fruits, such as citrus fruits, hawthorn berry, and also can be synthesized in accordance with the conventional process described by Seka, Prosche and Monatsh., 69,284 (1936) and Zemplen, Bognar in Ber., 1043 (1943), and EINECS 222-963-8, Journal of European Communities; June 1990.

For example, rutin may be found in hawthorn berries, flowers, leaves, stems, and roots in an amount ranging from 0.2 to 5 wt. % (PDR Herbal Medicines, 2nd Edition 2000). Rutin, quercetin, kaempherol and vitexin may be extracted from hawthorn berry by using a suitable solvent, such as water or aqueous ethanol alcohol under high temperature and pressure. The other method is using aqueous solution of ½ N of Ca (OH) sub.2 or NaOH, and then the crude extract and precipitates may be collected after neutralization. Furthermore, the dry powders of hawthorn berries, leaves, stems, flowers and roots may also be used. Generally, content of rutin, quercetin, kaempherol and vitexin in the berries is 5%, 3%, 2%, and 0.5% respectively.

Rutin, Quercetin, Kaempherol and Vitexin not only possess an inhibitory, but also exert a therapeutic effect on elevated plasma lipid level related diseases, such as hyperlipidemia, hypercholesterolemia, atherosclerosis, arteriosclerosis, stroke (cerebro-vascular accident), angina pectoris and hepatic disease, such as fatty liver and fatty degeneration. Furthermore, rutin, quercetin, kaempherol, and vitexin exhibit no toxicity and no adverse effects on hematopeutic, renal, hepatic systems when they are administered orally to a mouse at a dose of 1500 mg/kg, 1250 mg/kg, 1000 mg/kg, 500 mg/kg, respectively, which corresponds to an oral administered dose of 50 to 150 gm. of hawthorn berry extract for a person weighing 50 kg.

The present invention also provides a pharmaceutical composition for inhibiting the formation of fatty streak onto the arterial endothelial wall, which comprise hawthorn berry extract as an active ingredient plus pharmaceutically acceptable excipients, carriers or diluents.

The hawthorn berry extract of the present invention may be prepared in accordance with any conventional method by using suitable solvents, such as water or lower alcohol (ethanol) and an aqueous alkali or alkaline earth-metal hydroxide solution such as Ca (OH) sub.2 or NaOH solution. For example, 0.5 to 1 N of 1-10 liters of a solvent is added to 1 kg of dried hawthorn berry and the mixture is kept at a temperature ranging from 25 to 70 degrees C for a period ranging from 1 to 10 hours. The resulting extract is filtrated and concentrated to a formation of a concentrated hawthorn berry extract. For instance, if an aqueous alkali or alkaline earth metal hydroxide solution is used, the filtrate is adjusted to a PH ranging from 4.0 to 7.0 by adding an acid thereto. The resulting solution is kept at a temperature ranging from 5 to 20 degrees C for a period ranging from 5 to 30 hours. The precipitate is then dried to obtain a hawthorn berry extract. On the other hand, when ethanol is used as a solvent, 1 to 10 liters of 30% to 100% of solvent are added to 1 kg of the dried hawthorn berry, and the mixture is kept at a temperature ranging from 25 to 70 degrees C for a period ranging from 1 to 10 hours, then the resulting mixture is filtrated and concentrated to obtain a hawthorn berry extract.

The hawthorn berry powder may be used in the present invention in place of the hawthorn berry extract. The hawthorn berry powder may be prepared by lyophilizing or drying the solid materials from hawthorn berry according to a conventional method and powdering it to a particle size ranging from 50 to 250 mu.m.

A pharmaceutical formulation may be prepared in accordance with any of the conventional methods and procedures. In preparing the formulation, the active ingredient is preferably admixed or diluted with a carrier, or enclosed within a carrier, which may be in a form of a capsule, sachet, or other container. When the carrier serves as a diluent, it may be a solid, semisolid, or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

The pharmaceutical examples of suitable carriers, excipients and diluents are lactose, dextrose, sucrose, sorbitol, mannitol, starches, gum acacia, alginates, gelatin, calcium phosphate, calcium silicate, cellulose, methyl cellulose, micro-crystalline cellulose, polyvinylpyrrolidone, - water, methyl-hydroxy-benzoates, propyl-hydroxy-benzoates, talcum, magnesium stearate and mineral oil. The formulation may additionally include fillers, anti-agglutinating agents, flavoring agents, lubricating agents, wetting agents, emulsifiers, preservatives and the like. The pharmaceutical compositions of the invention may be formulated to provide quick, sustained or delayed release of the active ingredient after its administration to a mammal by employing any of the procedures and/or methods well known in the art.

The pharmaceutical composition of the present invention contains the active ingredient in an amount ranging from 0.01 to 100 mg/kg/day, but preferably from 0.1 to 50 mg/kg/day. It can be administered via various routes such as oral, transdermal, subcutaneous, intramuscular, intravenous, inhalational, intraperitoneal and transmucosal introduction. A typical daily dose of bio-flavonoids in human may range from 0.1 to 500 mg/kg of body weight, but preferably from 1.0 to 100 mg/kg of body weight and may be given in a single dose or in divided doses. The actual and exact amount of the active ingredient to be administered may vary according to patient's age, sex, body weight, disease, severity of illness and route of administration.

Furthermore, Quercetin, rutin, kaempherol and vitexin may be incorporated into foods and/or beverages for the purpose of preventing and/or treating elevated plasma lipid related diseases (e.g., hyperlipidemia, hypercholesterolemia, atherosclerosis, arteriosclerosis, cerebro-vascular accident, angina pectoris and hepatic disease). The foods and beverages may include food products, meats, vegetable juices, fruit juices, snacks, confectionery (chocolates and pizza), gum, dairy products, soups, broth, pastes, sauces (such as ketchup), teas, alcohol beverages, carbonated beverages, vitamin complexes and various health foods.

The content of rutin, quercetin, kaempherol and vitexin, or a mixture thereof in a food or beverage may range from 0.1 to 10 wt %. It is therefore comprised of 1 to 100 gm of rutin, quercetin, kaempherol, vitexin or mixture thereof per 1000 ml of beverage.

### EXAMPLE 1

### Preparation and Analysis of Hawthorn Berry Extract

The hawthorn berries (Ogden, Utah. USA) were dried at room temperature and powdered to a particle size ranging from 100 to 200 mu.m. and then 50 ml of 80% ethanol was added to 10 gm of hawthorn berry powder and extracted in a water bath at 60 degree C for 6 hours. The extract obtained was filtrated and cooled, then ethanol was added to the filtrate to a volume of 50 ml.

The above extract in an amount of 3.0 mu.l. was subjected to high performance liquid chromatography (HPLC) using prostar UV-Vis spectrophotometer lichrosorb RP-8 column (5 mu.m, 4 times, 250 mm) was pre-calibrated with 0.1 M borate sodium duodisulfate (SDS) solution and maintained at a temperature of 30 degree C. The extract was eluted with 0.1 M of borate SDS at flow rate of 0.5 ml/min. Standards solution were prepared by dissolving rutin, quercetin, kaempherol (Aldrich-Sigma Chemical Co. St. Louis, Mo. USA) and vitexin (Indofine Chemical Co. Somerville, N.J. USA) in 0.1 M borate SDS to a final concentration of 0.1, 0.2, 0.3, 0.4, and 0.5 mg/ml respectively, and subjected to HPLC under the same condition as that of above. The elutes were detected at 254 nm.(rutin), 257 nm.(quercetin), 266 nm.(Kaempherol), 270 nm.(vitexin) with UV-Vis spectrophotometer and the contents of rutin, quercetin, kaempherol and vitexin were calculated by comparing by the areas of HPLC profiles of the hawthorn berry extract and standard solution. The contents (%) of rutin, quercetin, kaempherol and vitexin in hawthorn berry extracts are depicted in table 1.
1. TABLE 1-Hawthorn berry extract contents Rutin 5.2%, Quercetin 3.1%, Kaempherol 2.3% and Vitexin 0.5%.

### EXAMPLE 2

### Preparation of Hawthorn Berry Extract

(A) Method of Using Ethanol

The hawthorn berries were dried at room temperature, 300 ml of 80% ethanol was added to 100 gm of dried berry, The berry were extracted at 60 degree C for 6 hours; the resulting extract was filtrated through cheese cloth and the filtrate was concentrated under vacuum to obtain 57 gm of syrupy extracts. The contents of rutin, quercetin, kaempherol and vitexin were examined in accordance with the method of example 1 which contained rutin 2.90 gm, quercetin 1.82 gm, kaempherol 1.31 gm, and vitexin 0.285 gm.

The composition of hawthorn berry extract was confirmed by HPLC and the result is depicted in Table II.
2. TABLE II-Ingredient content (%), moisture 67%, fructose 4%, glucose 3%, sucrose 2.9%, rutin 5.1%, quercetin 3.2%, kaempherol 2.3%, vitexin 0.5%, others 12.1%.

(B) Method of Using NaOH

The dried hawthorn berries (Ogden, Utah. USA) in an amount of 100 gm was added to ½ N 500 ml of NaOH solution, and kept at room temperature for 3 hours while stirring. The resulting extract was obtained by filtrating through a cheese cloth, then 1 N HCl solution was added to the filtrate to adjust its PH to 4.5. The same procedure as that of above was repeated to obtain a filtrate to adjust its PH to 6.5. The resulting filtrates were kept at 6 degree C for 12 hours and then the precipitates were collected and dried to obtain 8.8 gm and 9.8 gm powders, respectively. The compositions were confirmed by HPLC analysis which showed that hawthorn berry extracts contained rutin, (4.08 gm, 4.55 gm), quercetin (2.56 gm, 2.85 gm), kaempherol (1.84 gm, 2.05 gm), vitexin (0.40 gm, 0.45 gm) and the purity was 29.9% and 20%, respectively.

(C) Method of Using Ca (OH) sub.2

The dried hawthorn berry (Ogden, Utah. USA) in an amount of 100 gm was added to ½ N 500 ml of Ca(OH) sub.2 solution, and kept at room temperature for 3 hours while stirring. The resulting extract obtained by filtrating through a cheese cloth, and then 1 N HCl solution was added to the resulting filtrate to adjust its Ph to 4.5. The same procedure as that of above was repeated to obtain a filtrate and to adjust its Ph to 6.5. The resulting filtrates were kept at 6 degree C for 12 hours and then the precipitates were collected and dried to obtain 1 gm and 2 gm powders, respectively, HPLC analysis of the powders showed that hawthorn berry extracts contained rutin (0.464 gm, 0.928 gm), quercetin (0.29 gm, 0.58 gm), kaempherol (0.209 gm, 0.418 gm), vitexin (0.455 gm, 0.910 gm) and the purity was 60% and 63%, respectively.

### EXAMPLE 3

### Toxicity of Rutin, Quercetin, Kaempherol, and Vitexin in Mice by Oral Administration

24 specimens of 8 week old, with specific pathogen free, ICR female mice, each weighing from 25 to 30 gm, were divided into four groups (6 mice each) and were kept in separate cages under an environment of 23+_3 degree C, relative humidity of 45+_5%, and 12 light/12 dark photoperiod, fed with Harlan Teklad-2018 global rodent diet (18% protein)(Kaytee Co. Madison, Wis. USA) and water was sterilized to feed to the mice.

Rutin, quercetin, kaepherol (Aldrich-Sigma Co. St. Louis, Mo. USA) and vitexin (Indofine Co. Somerville, N.J., USA) were dissolved in 0.5% of tween-80 solution to a final concentration of 150 mg/ml, 12.5 mg/ml, 100 mg/ml and 50 mg/ml respectively, and was orally fed to the 4 separate groups of mice in an amount of 0.2 ml per 20 gm of mouse body weight, i.e., rutin 1500 mg/kg, quercetin 125 mg/kg, kaempherol 1000 mg/kg and vitexin 500 mg/kg respectively. The solution was administered once and the mice were observed for 180 days for signs of adverse effects or death according to the following schedule: 1H, 4H, 8H, 12H, (hour) after administration and then every 12 hours thereafter. The daily weight of each mouse was recorded. On day 181, the mice were sacrificed and the internal organs including liver, kidney, heart, lung, muscle, stomach, urinary bladder, intestines, pancreas and spleen were examined visually and microscopically.

All mice were alive at day 180 and no body weight loss occurred during this period of observation. The mice did not develop any pathological abnormality either visually or microscopically. Therefore, it is concluded that the hawthorn berry extract which includes rutin, quercetin, kaempherol and vitexin is not toxic when orally administered to a mammal.

### EXAMPLE 4

### Administration of Hawthorn Berry Biolavonoids; Rutin, Quercetin, Kaempherol and Vitexin to Rabbits

(Step A) 36 specimens of three month old New Zealand white rabbits (Harlan Kaytee Co. San Diego, Calif.) each weighing 2.5-to 2.6 kg were fed under a condition of temperature of 23+_3 degree C, relative humidity of 45+_5% and photoperiod 12 light/12 dark. The rabbits were divided into six groups with 6 heads each and were fed with six different diets; Harlan Taklad rabbit diet-TD-1376 (Madison, Wis. USA) which contained of 1% cholesterol in control group; 1% cholesterol plus 1.5 mg/kg Simvastatin (Merck Co. N.J. USA) in comparative group; 1% cholesterol plus 0.15% rutin in rutin group; 1% cholesterol plus 0.15% quercetrin in quercetin group; 1% cholesterol plus 0.15% kaempherol in kaempherol group; 1% cholesterol plus 0.15% vitexin in vitexin group.

Harlan Taklad rabbit diet TD-1376 contains moisture 12%, crude protein 16%, crude fat 2%, crude fiber 15%, ash 8%, and nitrogen free substances 47%.

The rabbits were fed for 8 weeks with free access to specific high cholesterol diets and water. Body weight was recorded every 7 days and the records were analyzed. All rabbits showed a normal growth rate and there were no significant differences among the six groups in regard to the diet ingestion amount and the body weight gain. (Rutin, quercetin and kaempherol were purchased from Aldrich-Sigma Co. St. Louis, Mo. USA) (Vitexin was purchased from Indofine Co. Sumerville, N.J. USA)

(Step B) After 8 weeks of breeding, the rabbits were anesthetized with injection of ketamine 75 mg/kg in the femoral muscle and sacrificed. Blood samples were collected from the heart of each rabbit to determine the blood analysis consisting of; complete blood count (CBC), Chemistry-7 and 24, (including Liver and renal function tests), lipid profiles, (including Total cholesterol, HDL, LDL, VLDL and triglycerides), coagulation factors consisting of; Prothrombin time (PT), Partial thromboplastin time (PTT), and immuno-globulin-E (an anti-allergenic factor).

### EXAMPLE 5

### Analysis of Plasma Total Cholesterol, HDL and Triglycerides in Rabbits

(Step C); The Effects of Administering Rutin, Quercetin, Kaempherol, and Vitexin to Rabbits on Plasma Cholesterol and Triglycerides Contents were Determined as Follows.

The blood samples collected from rabbits of the six dietary groups were allowed to stand for 2 hours, then centrifuged at 4000 rpm for 10 minutes. (Megafuge, Baxter-Heraeus instrument Co. N.J., USA) The supernatants were separated and stored in a deep freeze before analysis. The chemistry analysis was carried out by blood chemistry analyzer (Cobras-Integra-700, Roche Diagnostic Lab. Indiana, USA) to determine the changes in total cholesterol, HDL, LDL, triglycerides, liver function tests (such as SGOT, SGPT, G-GPT) and coagulation factors (PT, PTT) (Bayer-MLA-Electra-900 Automatic coagulation timer). The results were tested by using student t-test and Microsoft excel-7.0 program. The results are depicted in Table III.
3. TABLE III.

Blood analysis on rabbits fed on 6 different high cholesterol diets

Control group, Simvastatin group, Rutin group, Quercetin group, kaempherol group, vitexin group.
Control group; TC; (383.3+_55.2 mg/dl), TRG (165.6+_40.6 mg/dl), HDL (45.6+_22.4 mg/dl), SGOT (35+_6 u/l), SGPT (62.5+_6.5 u/l), GGTP (5+_2 u/l); WBC; (4.8+_2.0 k/ul), A; (33+_10), B;(3.5+_0.5).
Simvastatin group; TC;(277.3+_90.7), TRG; (141+_30), HDL; (47.5+_16.5), SGOT; (114.3+_30.7), SGPT; (71.2+_3.8), GGTP; (6+_1); WBC; (3.3+_2.6 k/ul), A; (13.5+_6.5), B; (2.8+_0.3).
Rutin group; TC; (254.5+_36.5), TRG; (108+_22), HDL; (36+_6), SGOT; (52.8+_12.2), SGPT; (33+_9), GGTP; (3+_1), WBC; (4.8+_1.2), A;(11.3+_6), B; (2.6+_0.4).
Quercetin group: TC; (262.3+_30.7), TRG; (105.6+_18.4), HDL; (42+_8), SGOT; (62+_7), SGPT; (43+_12),GGTP; (4+_1), WBC; (4.5+_1.5). A;(12.8+_5.4), B; (2.7+_0.3).
Kaempherol group; TC; (275+_23), TRG; (130+_26), HDL; (45.3+_7.3), SGOT(65+_8.6), SGPT; (42+_8.8), GGTP; (3.2+_1.8), WBC; (4.6+_1.8), A; (13.5+_4.6), B; (2.8+_0.4).
Vitexin group; TC; (269.5+_38.5), TRG; (138+_28), HDL; (46+_12), SGOT; (60+_12.5), SGPT; (48+_15), GGTP; (3+_1.5), WBC; (4.9+_1.7), A; (13.7+_4.3), B; (2.9+_0.5).

TC: Total Cholesterol, TRG: Triglycerides, WBC: White Blood Cell, HDL: high density lipoprotein, A: percentage (%) proportion of fatty streak to total aortic area, B: percentage (%) proportion of fat containing cells.

From the data of Table III, administration of rutin and quercetin, kaempherol and vitexin decreased plasma total cholesterol and triglycerides by 32-33% and 45-47%, also 30-30% and 22-17%, respectively, as compared to that of control group. Rutin, quercetin, kaempherol and vitexin are more effective in reducing plasma total cholesterol and triglycerides than Simvastatin, furthermore, liver function and WBC are not affected as that of the Simvastatin group.

(Step D) Analysis for Fatty Streak Deposition in Thoracic Aorta.

The chest wall of each rabbit (sacrificed in Step B) was incised, a portion of thoracic aorta from 1 cm site above aortic valve downward for 5 cm was cut out. The surrounding fatty tissues were removed, the aorta was incised longitudinally and pinned to a dish. The moist aorta was then photographed, and staining of fatty streak was carried out with the method described by Esper, E. et al. (Journal of Lab. Clinical Medicine; 121, pp 103-110(1993)) as follows.

The opened portion of aortas were pinned to a wooden tongue depressor, washed three times for 2 minutes with anhydrous propylene glycol and stained for 30 minutes with saturated solution of Oil Red 0 dissolved in propylene glycol. Then the aortas were washed twice for 3 minutes with 85% propylene glycol, to remove remaining staining solution by washing with normal saline solution. The aortas were photographed, and the photographs were traced with an image analyzer (LEICA, Q-600, Germany), and the area of stained portion were fatty streak region, its proportion in percentile (%) to the total aorta area were calculated. The results were shown in Table III.

FIGS. IA, IB, IC, ID, IE, IF show that the aortas of the rabbits administered with 1% cholesterol control group; 1% cholesterol plus 1.5 mg/kg simvastatin in comparative group; 1% cholesterol plus 0.15% rutin group; 1% cholesterol plus 0.15% quercetin group; 1% cholesterol plus 0.15% kaempherol group; 1% cholesterol plus 0.15% vitexin group respectively. The microscopic pictures which showed a thick layer of macrophage lipid cell complex in the arota walls of IA, but no or a very thin layer of macrophage lipid cell complex in the aorta walls of IB, IC, ID, IE, IF. (Pictures are not shown in this setting).

It is concluded that the rutin, quercetin, kaempherol and vitexin in the present invention can prevent and/or inhibit the deposition of macrophage-lipid cell complex onto arterial endothelial walls. Therefore, long term administration would prevent and inhibit atherosclerosis and arteriosclerosis induced cardiovascular diseases and the formation of fatty liver as depicted on Table III. The results were tested by using student t-test and Microsoft excel-7.0 program.

(Step E) Pathological Examination of Rabbit Organs.

Internal organs from the rabbits sacrificed in Step B including aorta, lung, heart, liver, kidney, muscle, bladder and pancreas were visually examined and showed no abnormalities. One half of each organ was frozen and the other half was fixed with 10% neutral buffered formalin solution for 24 hours. Then the fixed organs were washed with tap water and stepwise dehydrated with 70%, 80%, 90%, 100% ethanol, then embedded in paraffin by using Shandon, Histocentre-2 The embedded organ blocks were sectioned in 4 mu.m thickness with a microtome (McBain, M 820, American Optical Co. USA) and stained with hematoxylin and eosin stain (H. E. stain). Then the stained specimens were made transparent with xylene, and mounted with permount on micro slides. There were no pathological abnormalities or lesions under microscopic examination.

### EXAMPLE 6

### Inhibition and Prevention of Fatty Degeneration in the Liver of the Rabbits.

According to the report of Fogt F. and Nanji A., (Toxicology and Applied Pharmacology; 136, pp 87-93, 1996) and Keegan A. et al., (Journal of Hepatology; 23, pp 591-600, 1995). Fatty degeneration of liver can be classified into four grades based upon the abnormal fat containing cells around the central vein of liver acinus, for example: grade 1 (0-25%), grade 2 (26-50%), grade 3 (51-75%), grade4 (76-100%). The effects of rutin, quercetin, kaempherol and vitexin on liver tissue on the rabbits from Example 4 Step B were examined. The results are depicted in Table III.

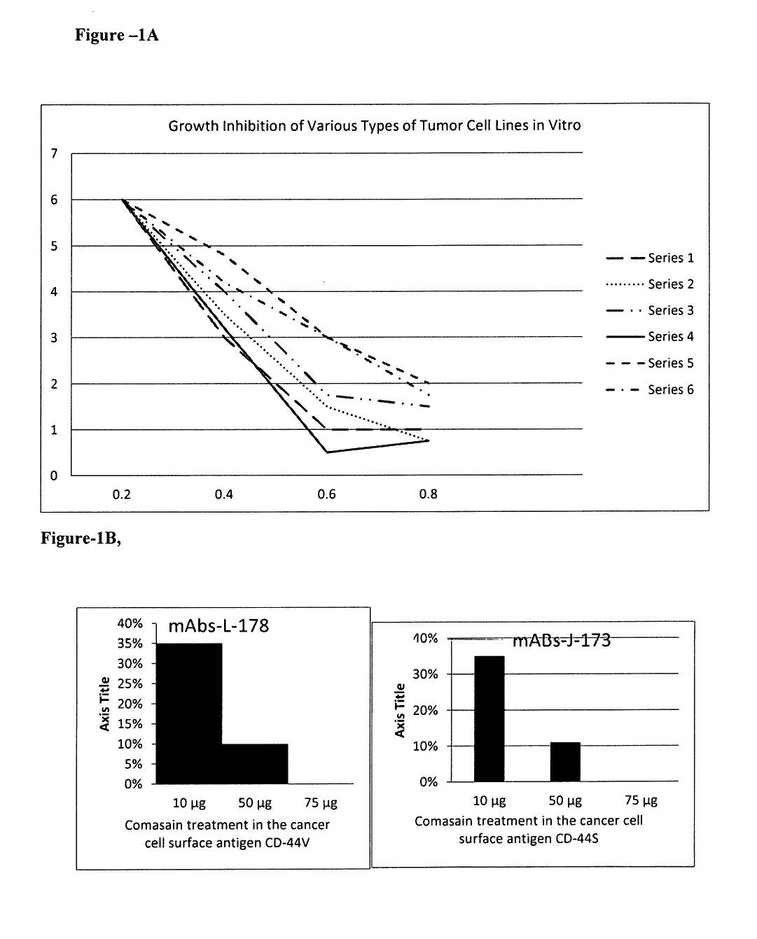

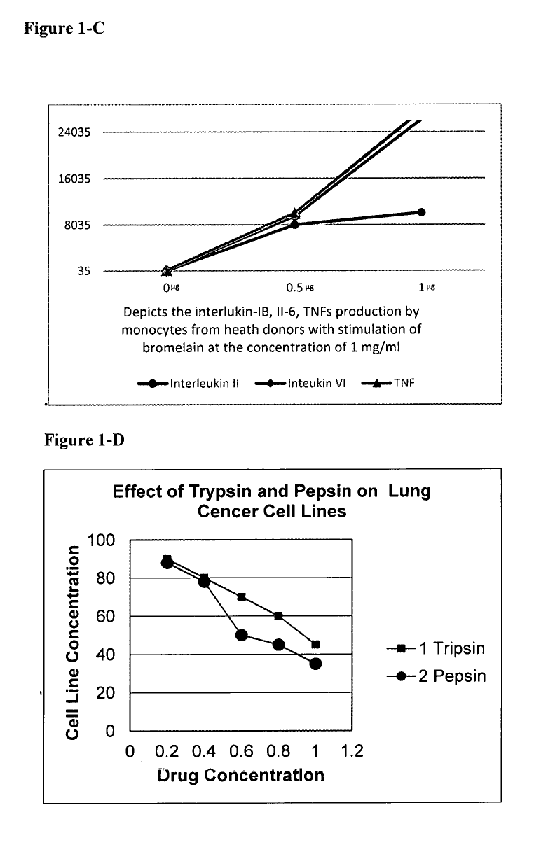

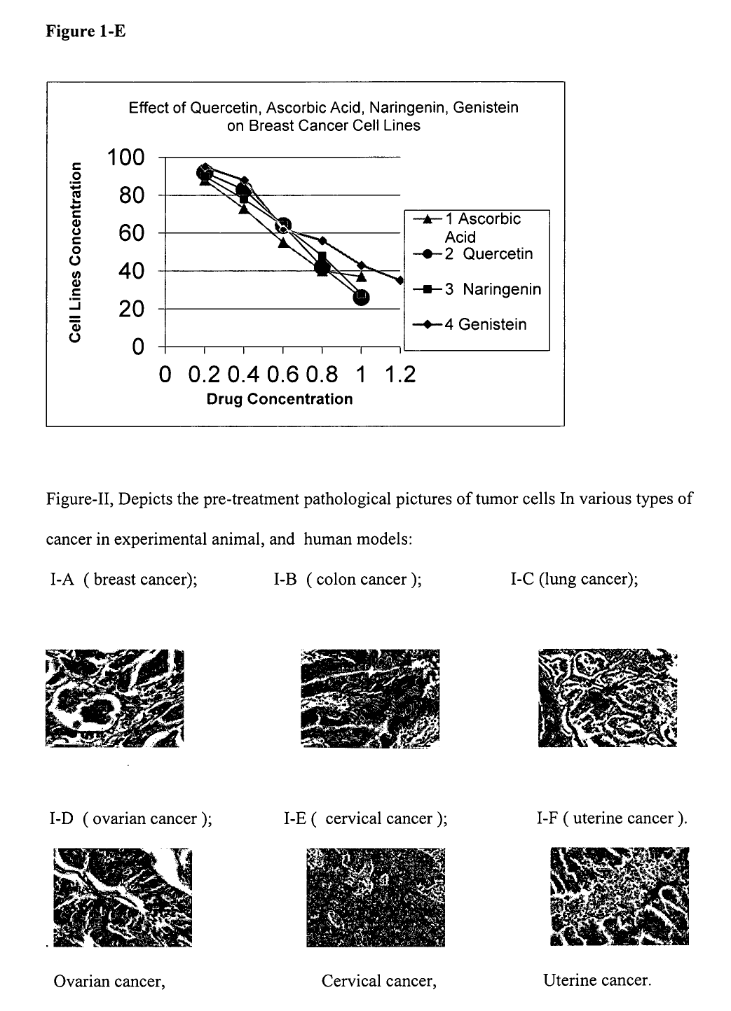

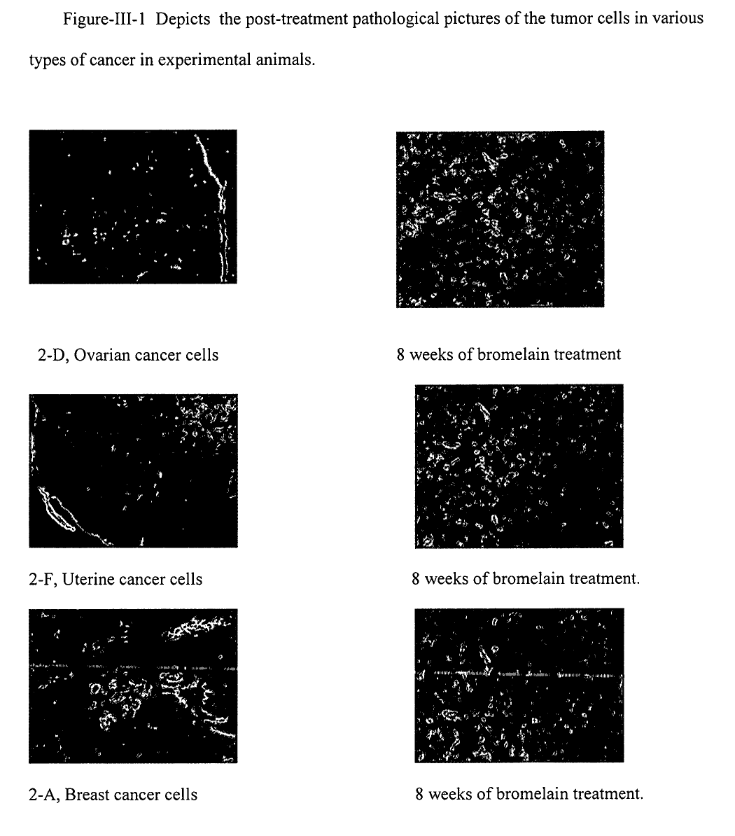

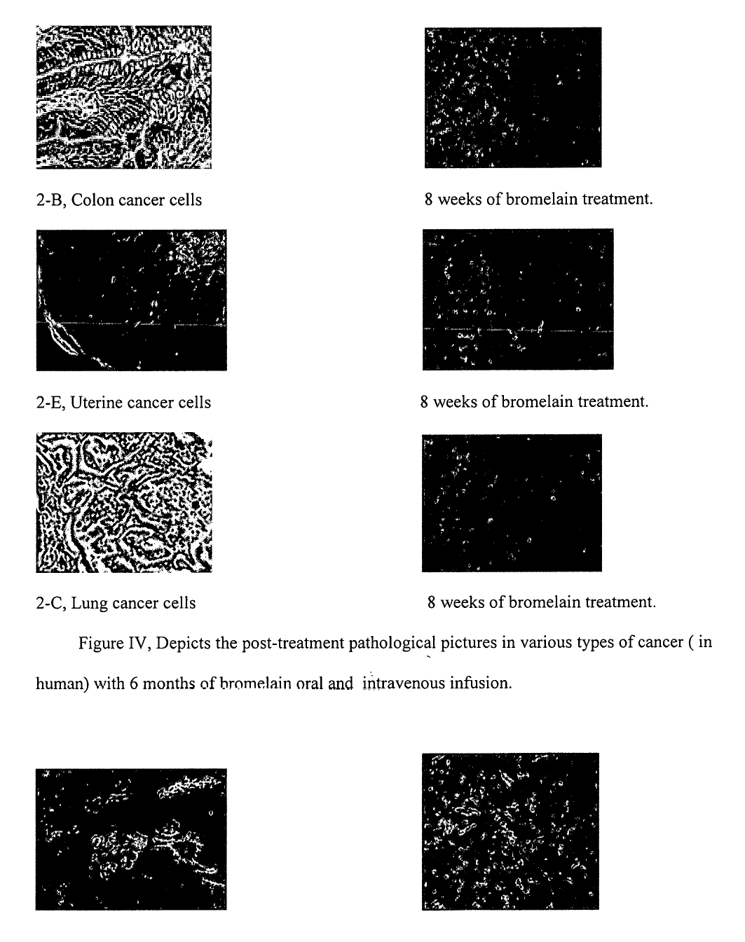

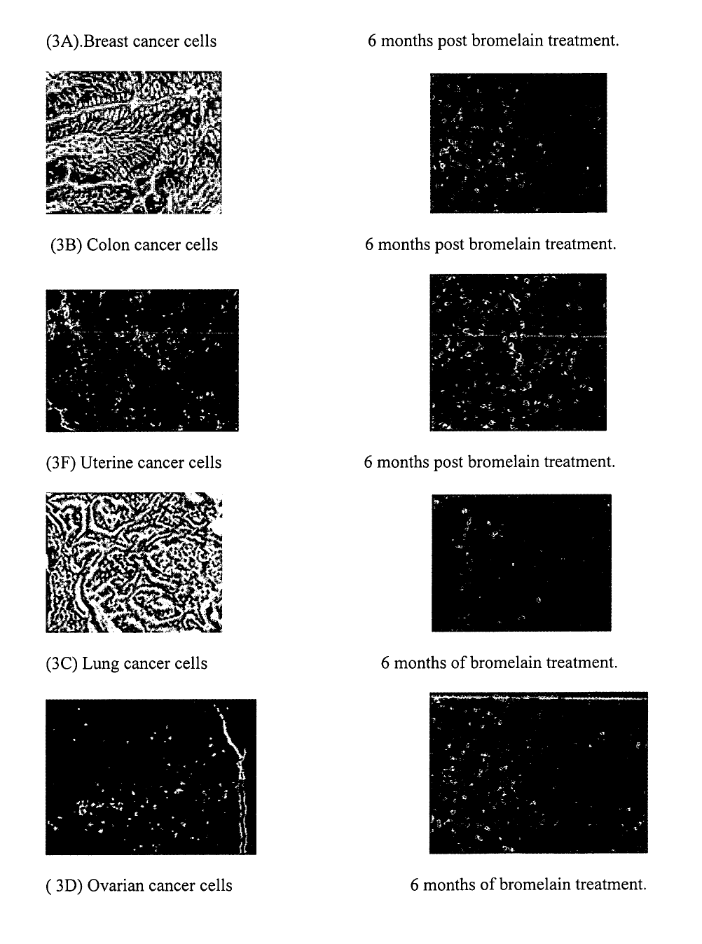

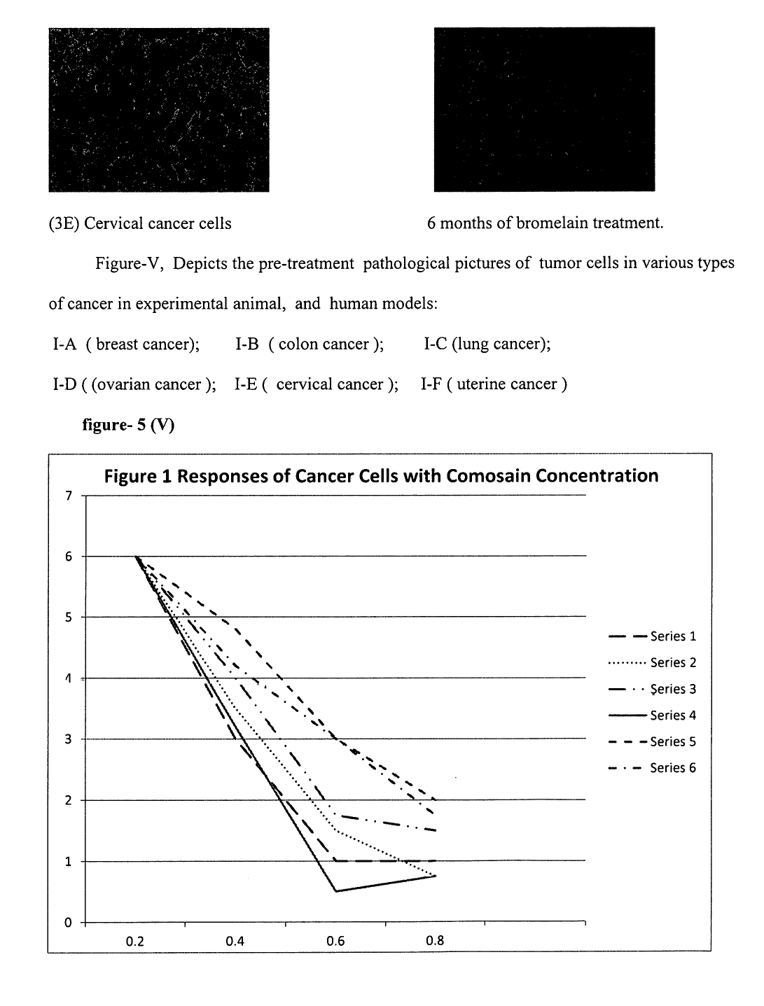

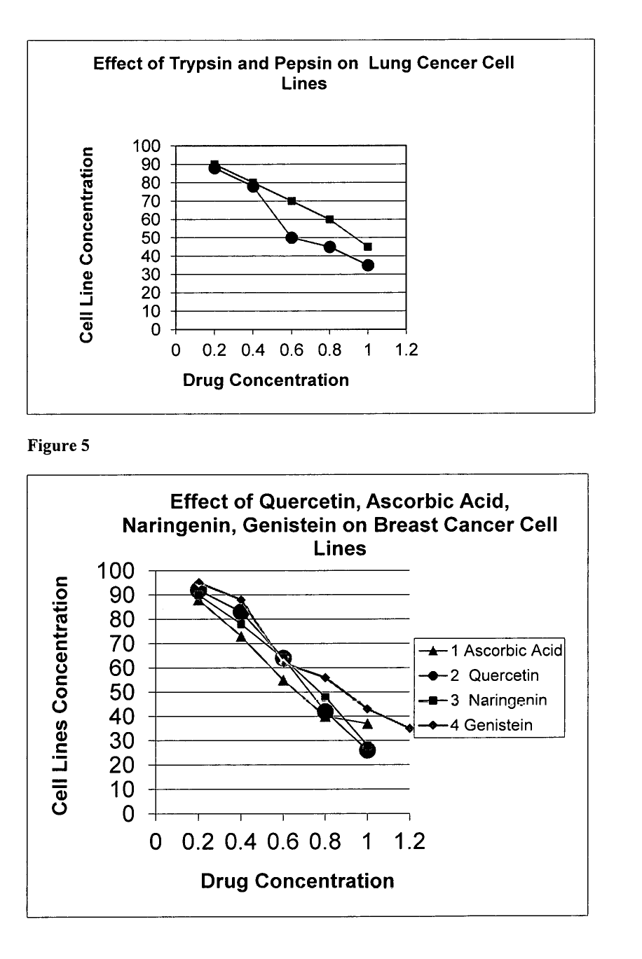

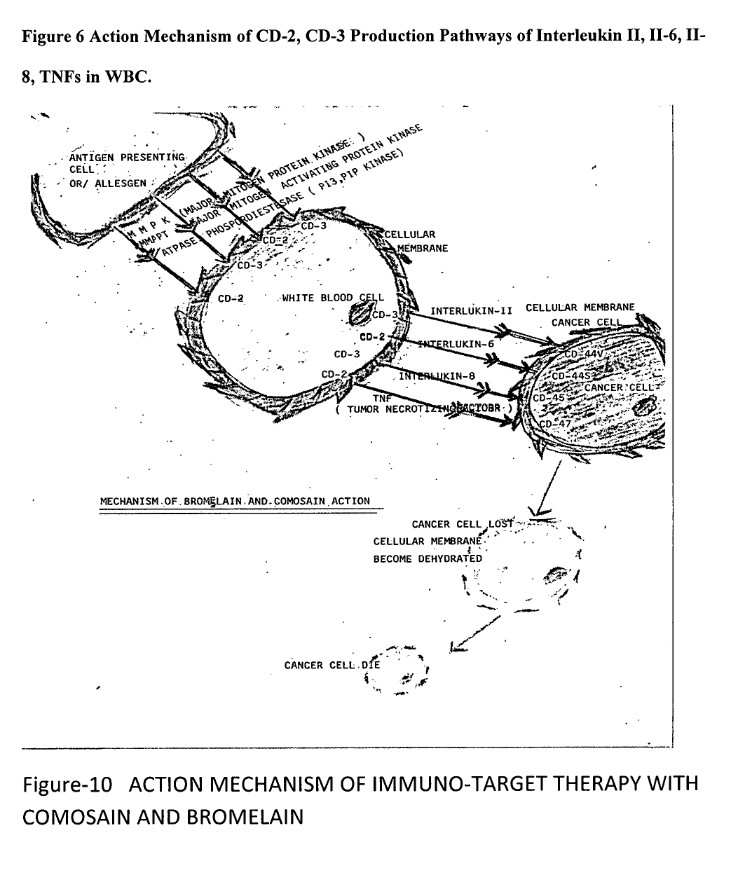

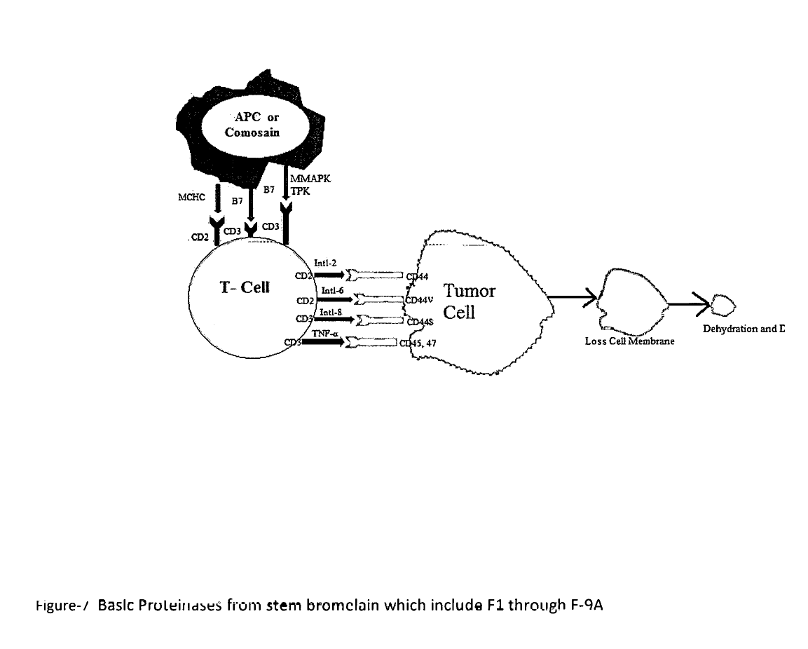

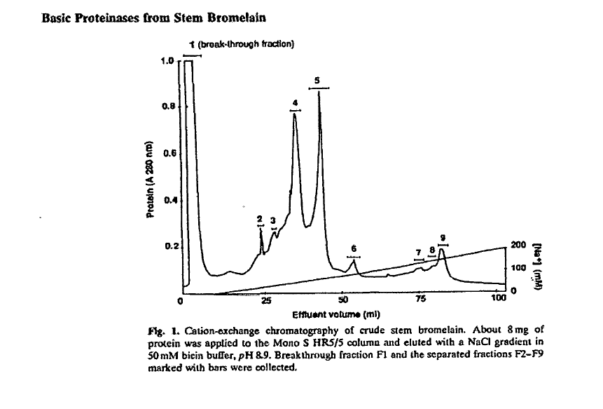

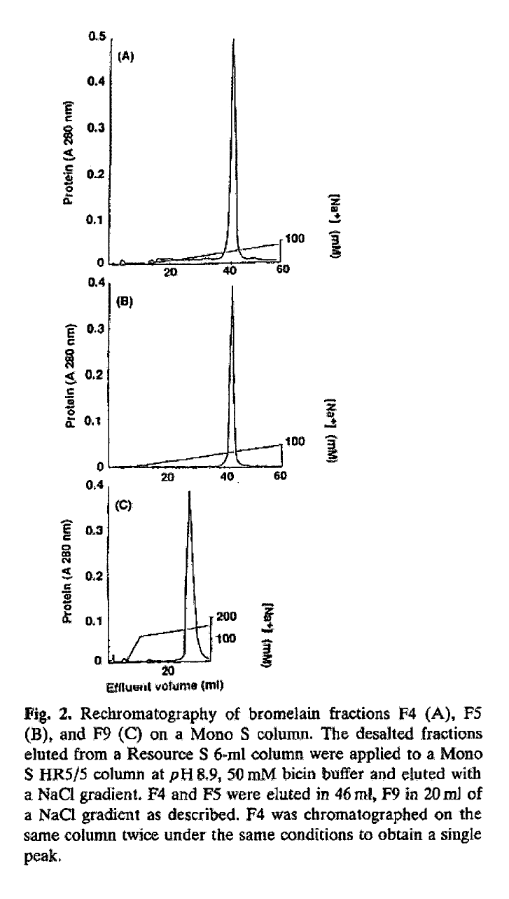

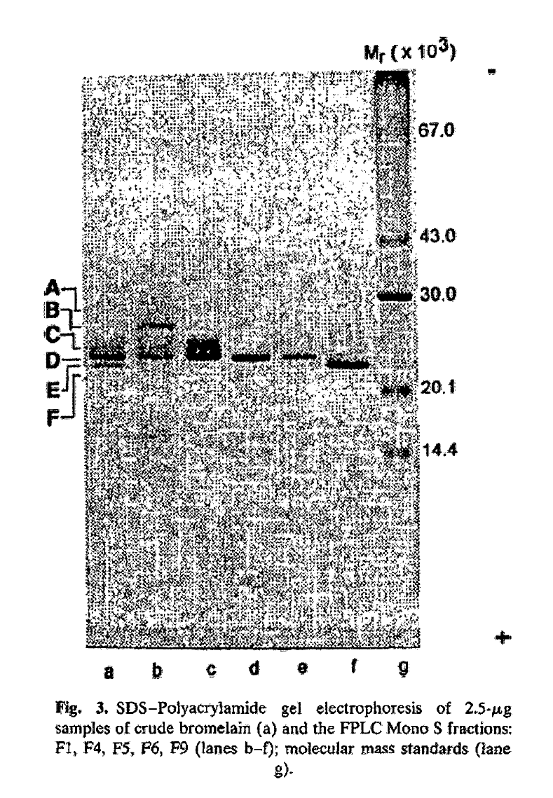

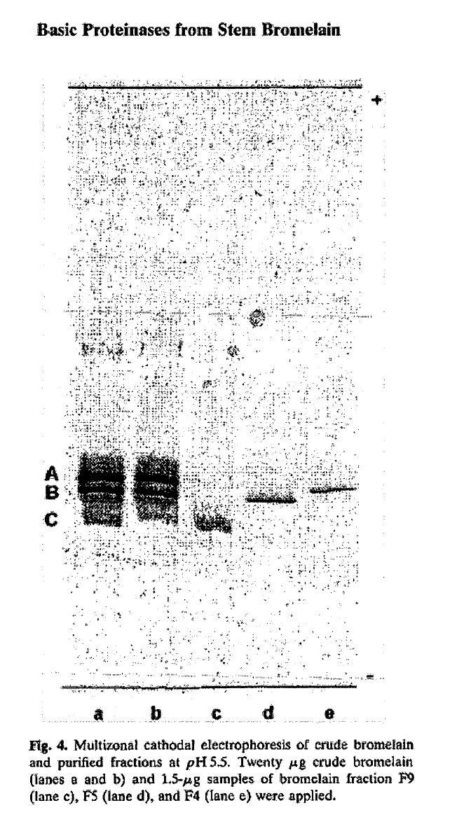

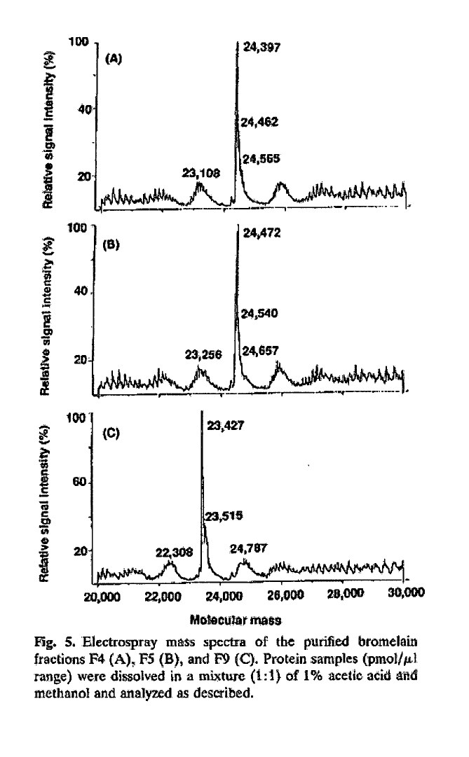

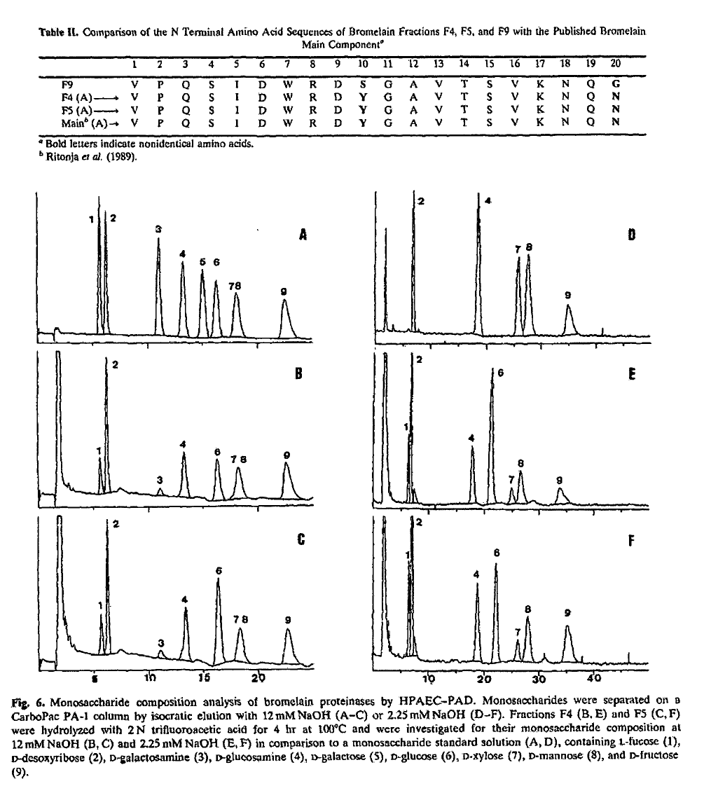

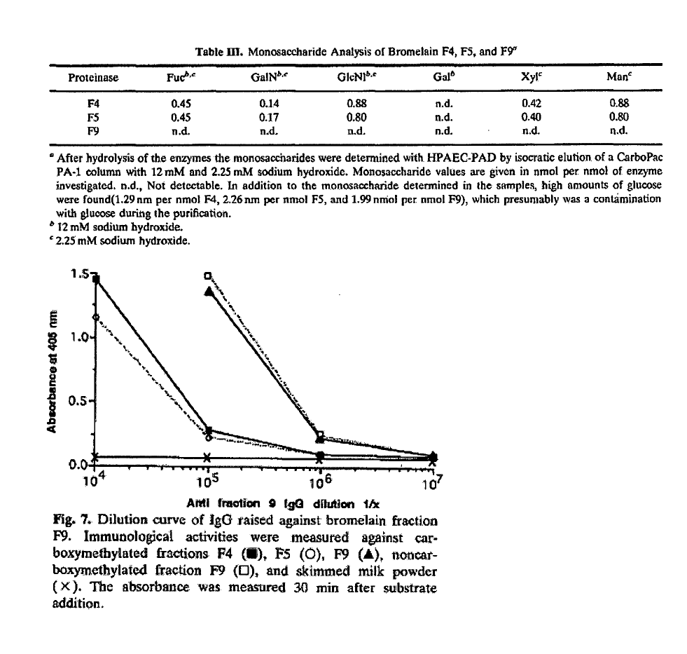

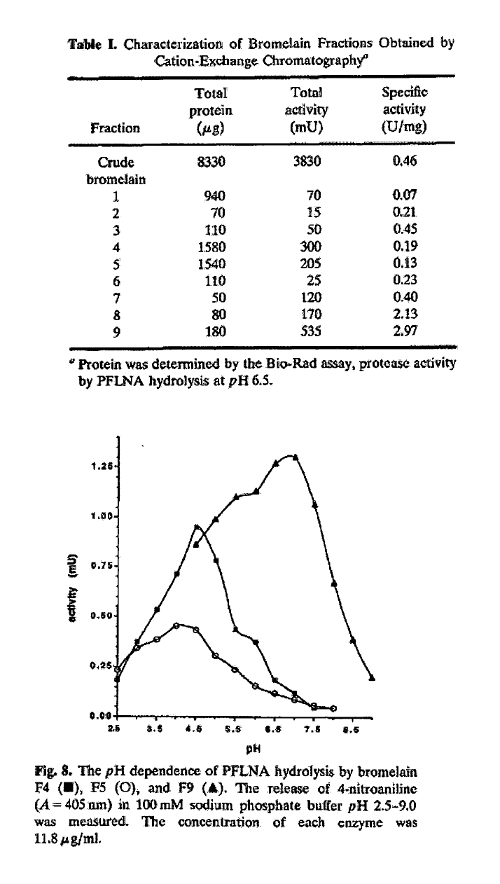

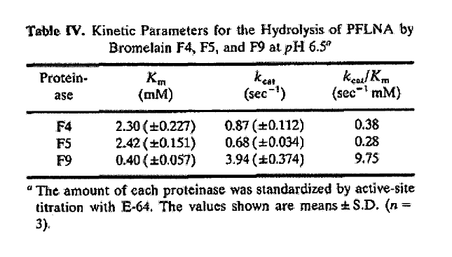

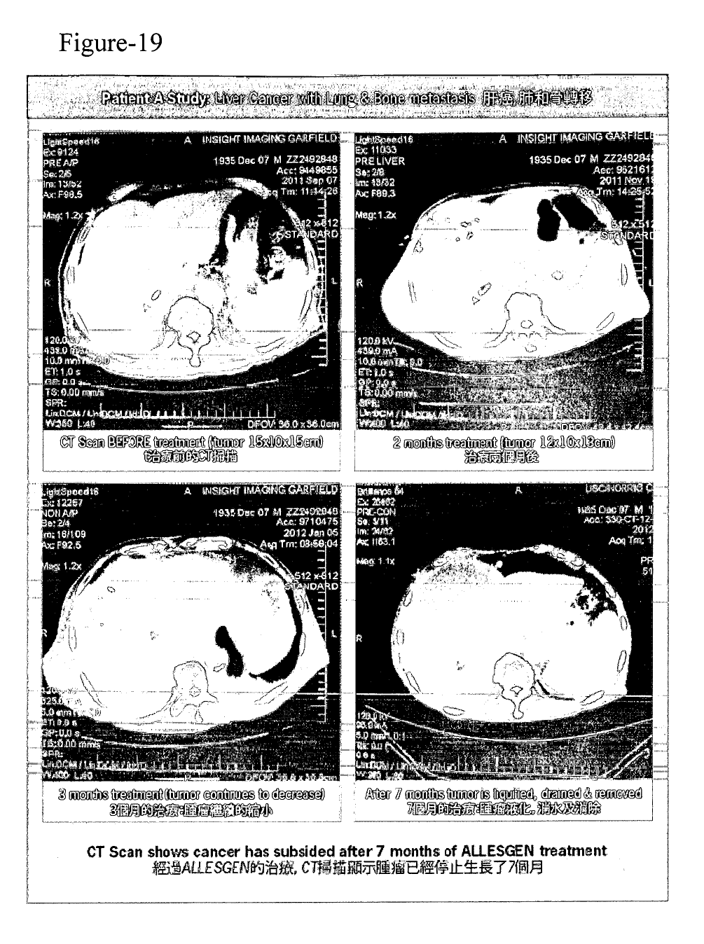

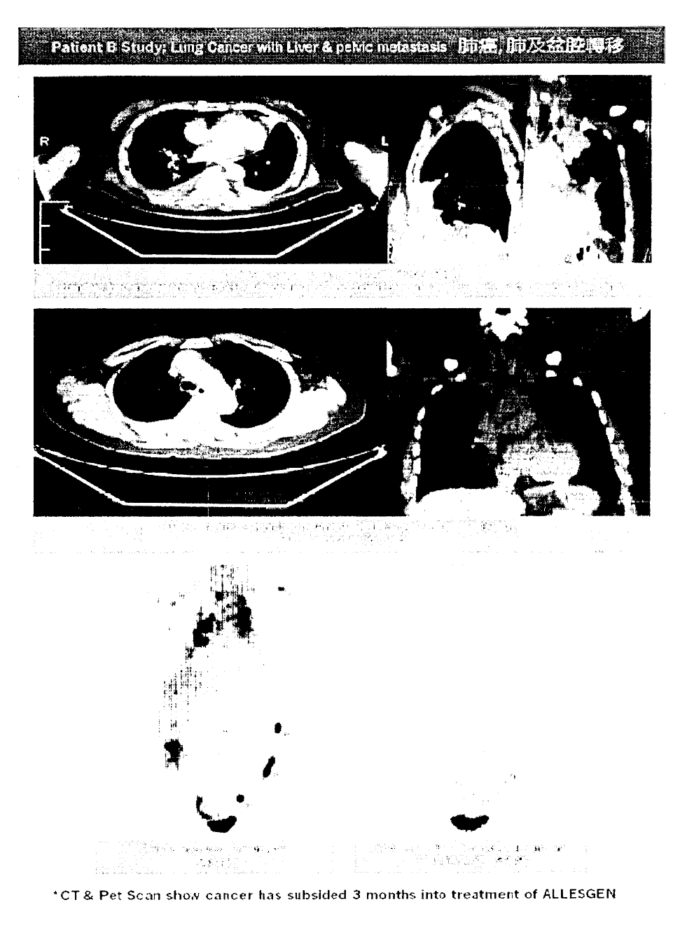

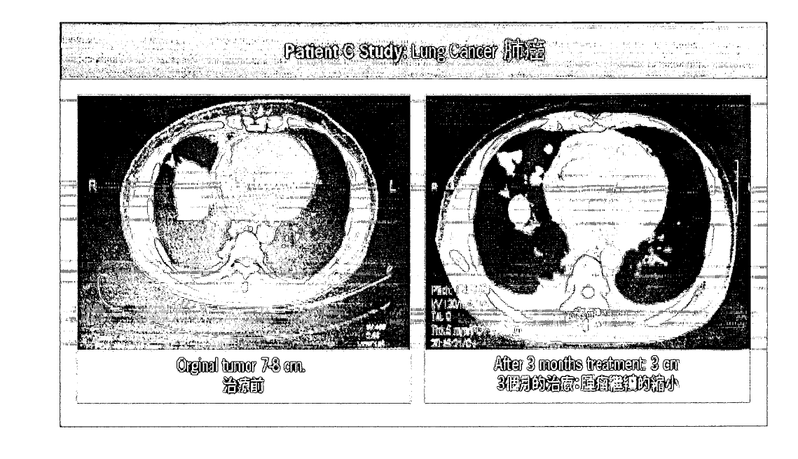

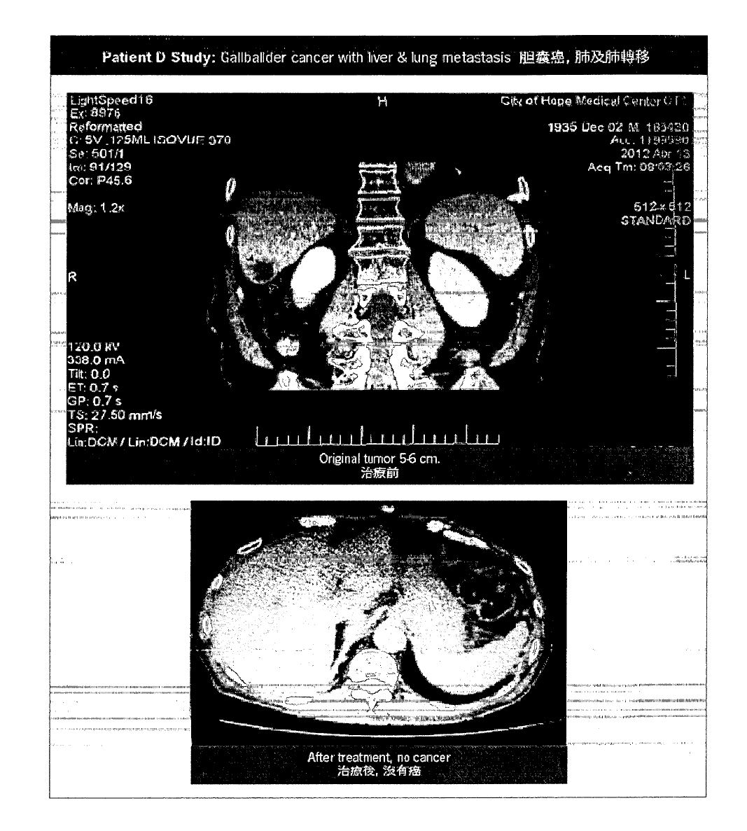

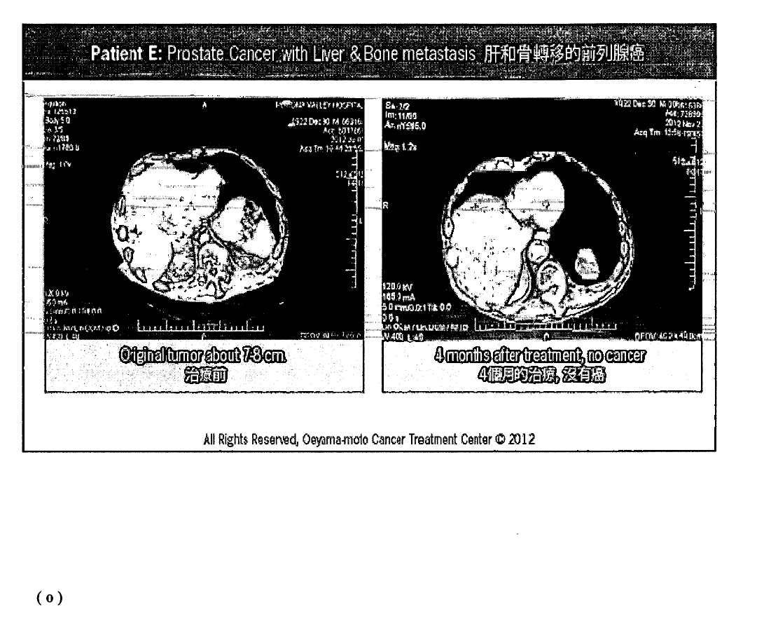

FIGS. 2A, 2B, 2C, 2D, 2E and 2F represent the microscopic features of the liver of the rabbits administered with 1% cholesterol; 1% cholesterol plus 1.5 mg/kg Simvastatin; 1% cholesterol plus 0.15% rutin; 1% cholesterol plus 0.15% quercetin; 1% cholesterol plus 0.15% kaempherol and 1% cholesterol plus 0.15% vitexin respectively. The features of 2A and 2B show many fat containing cells around the central vein. The other features in 2C, 2D, 2E and 2F showed that almost all of the liver cells are normal without containing fat particles. It depicts and shows that rutin, quercetin, kaempherol and vitexin can inhibit and prevent formation of fatty liver and fatty degeneration. Furthermore, one of 2B contains hepatic adenoma picture. This depicted that Simvastatin has serious side effect to the liver even with short term administration.( all the pictures are not shown in this settins)

### EXAMPLE 7

### HMG-CoA Reductase Inhibited by Rutin, Quercetin, Kaempherol, and Vitexin

The activity of 3-HMG-CoA reductase was determined by modified Hulcher's method (Journal of lipid research; 14, 625-641, 1973), in which the concentration of Co-A will be produced when 3-HMG-CoA is reduced to mevalonate salt by the action of the 3-HMG-CoA reductase; then the reductase was determined by spectroscopy and the activity of the 3-HMG-CoA reductase was calculated.

FIG.-I, Depicts the pre-treatment pathological pictures of tumor cells In various types of cancer in experimental animal, and human models:

| | | |
|---|---|---|
| I-A (breast cancer); | I-B (colon cancer); | I-C (lung cancer); |
| I-D (Ovarian cancer) | I-E (Cervical cancer) | I-F (Uterine cancer) |

FIG.-II, Depicts the post-treatment pathological pictures of the tumor cells in various types of cancer in experimental animal models which were treated with intra-peritoneal injection of bromelain In an amount of 25 mg/twice a week for 8 weeks which all shown complete resolved of all 6 types of cancer.

FIG. III, Depicts the post-treatment by either CT scan report and/or pathological pictures in various types of cancer in human model which were treated with bromelain by oral and intravenous infusion. All tumors were either completed resolved or more than 50% resolved

### DETAILED DESCRIPTION OF THE INVENTION

### DETAILED DESCRIPTION OF THE PREFERRED EMBODIMENTS

Bromelain extract including both stem and fruit bromelains, have all the effects that natural bromelains depict including anti-inflammation, anti-platelet aggregation, fibrinolytic properties, anti-tumor growth, and differentiation effect in tumor cells. Fruit bromelain from pineapple was first isolated by

Marcano in 1891 (Marcino Bull. Pharm. 5, 77 (1891), and from pineapple juice by precipitation with acctone and also with ammonium sulfide: Heinecke in 1961 (U.S. Pat. No. 3,002,891). Bromelain has a molecular weight of 33,000. It is a glycoprotein, was purified from crude preparations by Gibian, and Bratfisch et al in 1960 (U.S. Pat. No. 2,950,227).

Stem bromelain was discovered by Balls et al in 1941, (Ind. Eng. Chem. 33; 950, 1941), both fruit and stem bromelains are acidic and basic proteins with ultrviolet light wavelength of 280 nm (A 1%/1cm 20.1)

### Preparation and Analysis of Bromelain (Comosain) Extract.

EXAMPLE-A

### Cloning and Expression of the Minigene of Comosain and its Extraction Procedure

### Assembly of Expression Vector for the Comosain Gene and Preparation of Genetically Modified Organism (GMO)

The oligo-deoxyribonucleotide probe synthesis was used, the phosphoramidite method was used for oligonucleotide synthesis. Each probe mixture contained a pool of 20-oligonucleotides sequences were:

Probe mixture: CoM-V sequence equivalium to: valine-proline-glutamine-serine-isoleucine,
aspartic acid-trytophan-arginine-asparagine-tyrosine-glysine-alanine-valine-threonine-
serine-valine-lysine-asparagine-glutarmine-glycine.
Probe mixture: CoM-V =
Val-Pro-Glu-Ser-Iso-Asp-Trp-Arg-Asn-Tyr-Gly-Ala-Val-Thr-Ser-Val-Lys-Asn-Glu-Gly
3′ CAA, GGA, GTT, --------------------------------------------------------, TTG, GTT, TT5′
V D G V
P W A K
Q R V N
S N T Q
I Y S G
V = Valine, P = Proline, Q = Glutamine, S = Serine, I = Isoleucine.
D = Aspartic acid, W = Tryptophan, R = Arginine, N = Asparagine, Y = Tyrosine.
G = Glycine, A = Alanine, V = Valine, T = Threoninie, S = Serine.
V = Valine, K = Lysine, N = Asparagine, Q = Glutamine, G = Glycine.

The probe mixtures were labeled at the 5′ end with [r³²p] ATP, 7500-8000 Ci/mmol (ICN) (1 Ci=37 GBq), by using T4 polynucleotide kinase.

Hybridization procedures: phage plaques were ampilified according to the procedures of Woo, except that GeneScreen Plus filters and NZYAM plates [NACL, 5 g; MgCl₂-6H20, 2 g; NZ-Amine A, 10 g; yeast extract, 5 g; Casamino acids, 2 g; maltose, 2 g; and agar, 15 g (per liter) were used, Phage particles were disrupted and the DNAₛ were fixed on filters (50,000 plaques per 8.4×8.4 cm filter). The air dried filters were baked at 80° C. for 1 hour and then subjected to proteinase K digestion [50 ug of proteinase k per ml of buffer solution containing 0.1M Tris-HCl (PH 8.0), 0.15 M NaCl, 10 mM EDTA, and 0.2% NaDodSo4 for 30 min at 55° C. Prehybridization with a 1 M NaCl/1% NaDodSo4 solution was carried out again at 55° C. for 4 hours or longer

The hybridition buffer contahined 0.025 pmol/ml of each of the 20 probe sequences of 0.9M NaCl/5 mM EDTA/50 m M solution phosphate, PH 6.5/0.5% Na Dod So4/100 ug of yeast tRNA per ml. Hybridization was carried out at 48° C. for 20 hrs by using the ComV probe mixture. (that is 2° C. below the lowest calculated dissociation temperature (td) for members of The mixture. At the completion of hybridization, the filters were washed three times with 0.9 M NaCl/90 mM sodium citrate, pH 7.0/0.1%NaDodSo4 at room temperature at hybridization and 10 min per wash.

For direct expression of the genomic Comosain gene, 4.8 kilobase (kb) of BstTy/Se-and BamAs/GL fragment of Comosain, which contains the entire gene. After converting the BstTy/Se (tyrosine-serine 10 amino acids)site into Bst As/GL(asparagine-to-glycine 20 amino acids) site with a synthetic linker(pBR322 ori) the fragment was insert into the unique BamAs/GL site of the expression vector pDSVL, which contains a dihydrofolate reductase (DHFR) minigene. The resulting Plasmid DSVL-gPlCOS (gene Plant Comosain) was then used to transfect New Zealand white rabbit ovarian (NWRO) cells by the calcium phosphate microprecipitate method. The transformants were selected by the medium lacking hypoxanthine and thymidine. The culture medium used was Dulbecco's modified Eagle's medium supplemented with 10% fetal calf serum, penicillin, streptomycin, and glutamine.

Isolation of Comosain mRNA.: The 4.8 kilobase Bst ty/se-Bam As/GL restriction fragment from Comosain was inserted into shuttle vector, pSV4ST. The resulting chimeric plasmid pSV gPLComo was used to transfect Cos-1 cells by the calcium phosphate micoprecipitate method. After culture for 72 hrs, RNA was prepared from the transected cells by the guanidinium thiocyanate procedure of Chirgwin et al. and poly(A)+ mRNA was isolated by binding to oligo-cellulose.(Aviv & Leder).

cDNA cloning.: A Comosain cDNA bank was constructed according to a modification of the general procedures of Okayama and Berg by using the poly(A)+ mRNA described above (Mol. cell biology 2, 161-170, 1982).

DNA sequencing.: Restriction fragments were cloned into M 13 phage vectors by using Escherichia coli strains JM 103 and/or JM 109 as host. (Messing, J. of methods enzymology 1983) and were sequenced by the dideoxy method of Sanger et al. some regions were sequenced by kinase labeling or end-fill labeling of restriction fragments followed by chemical cleavage as described by Maxam and Gilbert. (J. of methods of enzymology 1980).

The final Comosain Recombinants were collected, extracted, washed with ethyl alcohol, and purified by AKAT Prime (GE Co.) and/or FPLC-cation-exchange chromatography, to produce of F4, F5, and F-9a, F9b (Comosain and Ananase) for future use.

EXAMPLE-B (see 68-B) Direct Expression of the Genomic Comosain Gene

EXAMPLE-C (see 68-C) Isolation of the Comosain m-RNA

EXAMPLE-D (see 68-D) Cloning of Comosain c-DNA.

### EXAMPLE-1

### Toxicity of Bromelain (Comosain) in Mice by Oral Administration

30 specimens of 8 week old, with specific pathogen free, ICR female mice, each weighing 25-30 gram, were divided into five groups (6 mice each) and were kept in separated cages under an environment of 23+_3 degree C, relative humidity of 45+_5%, and 12 H light/12 H dark photoperiod, they were fed with Harlan Teklad-2018 global rodent diet (18% protein) (Kaytee Co. Madison, Wis.) ; drinking water was sterilized.

Bromelain (Comosain) was dissolved in 0.5% of tween-80 solution to a final concentration of 50 mg/ml, 100 mg/ml, 125 mg/ml and 150 mg/ml respectively, and was orally fed to the 4 separated groups of mice in an amount of 0.2 ml per 20 gram of mouse body weight, that is contains 500 mg/kg, 1000 mg/kg, 1250 mg/kg, and 1500 mg/kg separately. One group of 6 mice was kept as control group and was not fed the bromelain solution. The solution was administered once every 2 weeks, and the mice were observed for 6 months for the signs of adverse effects or death according the following schedule: 1 H, 4 H, 8 H, 12 H (hour), after administration and then every 12 hours thereafter. The daily weight of each mouse was recorded. On day 80, (6 months later) the mice were sacrificed; and the internal organs including liver, kidney, heart, lung, muscle, stomach, urinary bladder, intestines, pancreas, and spleen were examined visually and microscopically.

All mice were alive at 6 months, and no body weigh loss occurred during this period of observation. The mice did not develop any pathological abnormality either visually or microscopically. Therefore, it is concluded that Bromelain extract from pineapple is not toxic when orally administered to a mammal.

### EXAMPLE-2

### Bromelain and Quercetin Inhibit Cancer Cell Growth In Vitro

Cancer cell lines were developed either from direct harvest from surgical specimens during surgery or from friends at different oncology institutes. The specimens were emulsified in normal saline and filtrated three times with the mesh permitting less than 5 um particles to pass through. The supernatants were preserved in Complete Growth Medium (CGM) (Sigma-Aldrich Co. St. Louis, Mo.) in the 75 ml flasks at 8 degree C until they are ready to be used for seeding in a standard tissue culture. Complete Growth Medium (CGM) consisting of: Dulbecco's modified Essential medium (Sigma-Aldrich Co. St. Louis, Mo.) supplemented with 10% heat inactivated new born calf serum, and 2 % L-glutamine, penicillin (100 IU/ml), streptomycin (5 mg/ml), and neomycin (10 mg/ml) (all from Sigma-Aldrich Co. St. Louis, Mo.) and incubated at 37 degree C with humidified atmosphere of 90% air, 10% CO2 and cell split at 1: 2 rate twice weekly.

Six various types of cancer cell lines which included breast, lung, colon, cervical, uterine, and ovarian cancer were used to test the sensitivity of the growth inhibition by the Bromelain in different concentrations. The cells were seeded in 2×(10)³ into 96 wells tissue culture microtiter plates (Becton-Dickinson Co. Franklin Lakes, N.J.). The cells were maintained in a standard tissue culture In Complete Growth Medium (CGM). Bromelain solutions were added to culture media in the following concentration: 0.2 mg/ml, 0.4 mg/ml, 0.6 mg/ml, and 0.8 mg/ml for 72 hours and tumor cells were counted with Coulter counter (Model B, Beckman Coulter, Co.). The tumor cell growth inhibition percentages are depicted in FIG.-2. which demonstrates that the growth of all cancer cell lines were inhibited in higher concentrations of Bromelain (Comosain).

### EXAMPLE-3

### Bromelain and Quercetin Inhibit Cancer Cell Growth In Vivo on Experimental Animal

### Bromelain and Quercetin Intraperitoneal Administration to Experimental Animal

14 Experimental animals of 4 to 6 weeks old white rabbits each weighting 1 to 1½ pound were fed under a condition of 23+_3 degree C, relative humidity of 45+_5% and photoperiod of 12 light/12 dark. The rabbits were divided into seven groups with 2 heads each and were fed with Harlan-Taklad rabbit die TD-1376 (Madison, Wis.) containing moisture 12%, crude protein 16%, crude fat 2%, crude fiber 15%, ash 8%, nitrogen free substances 47%.

The rabbits were fed for 3 weeks with free access to the diet and water body weight was recorded every 7 days, and records were analyzed. All rabbits showed a normal growth rate with no significant differences among the seven groups in regard to the diet ingestion amount or the body weight gain. The cancer cell lines were injected into six groups of the rabbits, that is 2 heads each group with 2 heads served as control (without tumor cells injection). The cancer cell lines were developed from Example-2. Each head was injected 0.5 ml of different cell line fluid intraperitoneally, prefer in the peritoneum layer, then the rabbits were fed the same diet for 3-4 weeks until a tumor grew in the peritoneum. The size, and location of the tumors were recorded. When the tumors reached to 3-5 mm diameter in size. Bromelain in the amount of 25.0 mg/ml in normal saline Quercetin 5 mg/ml with 100 mg of vitamin C (to keep the solution acidified), 2 ml of Bromelain was injected into six different group of rabbits, the bromelain were given twice a week for 8 weeks.

After 8 weeks of treatment, the rabbits were anesthetized with injections of ketamine 75 mg/kg in the femoral muscle and sacrificed. Blood samples were collected from the heart of each rabbit to determine the blood analysis consisting of: complete blood count (CBC), Chemistry-7 and 24, (including Liver and renal function tests), lipid profiles (including Total cholesterol, HDL, LDL, VLDL, and triglycerides), coagulation factors consisting of; prothrombin time (PT), partial thromboplastin time (PPT), and immune-globulin-E. All the laboratory tests were analyzed, and showed no differences among or within each groups. All laboratory blood analysis were performed on rabbits of all 7 groups, The results were tested using student t-test and Microsoft Excel-7 programs. The results are depicted in Table-II

Table-II, The table presents blood analysis of the rabbits of 6 different groups which were treated with bromelain after inoculation of cancer cell lines. Control group; TC (183.3+_50.2 mg/dl), TRG (110+_40.6 mg/dl), HDL (45.6+_20.4 mg/dl), SGOT (38.6+_6.2 u/l), SGPT (62.5+_6.5 u/l), GGTP (8+_2.4 u/l), WBC (6.8+_2.0 k/ul), Hb; (12.3+_2.2 gm/dl). Bromelain treated groups; TC (175.6+_36.8 mg/dl), TRG (92.6+_38.8 mg/dl),HDL (43.6+_16.5 mg/), SGOT (110.8 +_30.7 u/l), SGPT (71.2+_3.8 u/l), GGTP (7+_1), WBC (7.3+_2.2 k/ul), Hb; (11.9+_1.9 gm/dl). TC; Total Cholesterol, TRG: Triglycerides, WBC: White Blood Cell, HDL; High Density Lipoprotein, SGOT; Serum Glutamo-Oxalic Transferase, SGPT; Serum Glutamo-Pyruvic Transferase, Hb; Hemoglobin.

The internal organs from the rabbits sacrificed in this sample including lung, heart, liver, kidney, muscle, omentum, intestine, stomach bladder and pancreas were visual examined and showed no abnormalities. One half of each organ was frozen and the other half was fixed with 10% neutral buffered formalin for 24 hours. Then the fixed organs were washed with tap water and stepwise dehydrated with 70%, 80%, 90%, 100% ethanol and then embedded in paraffin by using Shandon- Histocentre-2. The embedded organ blocks were sectioned in 4 mu.m thickness with a microtome (McBain, M 820, American Optical Co. USA) and stained with hematoxylin and eosin stain (H.E. stain). The stained specimens were made transparent with xylene, and mounted with permount on microslides. There were no pathological abnormalities or lesions under microscopic examination. All the specimens collected from six group of rabbits showed no evidence of persistent disease or cancer cells. Therefore, we conclude that Bromelain can served as a chemotherapeutic agent in various types of cancer in experimental animal without side effects.

### EXAMPLE 4

### Bromelainase (Comosain) and Quercetin Oral Administration to Late Stage Center Patients to Inhibit Tumor Growth

24 volunteers were divided into six groups (four in each group) and 6 persons serve as control group (no bromelain treatment). All were in their 4th and 6th decades with various types of cancers including breast, lung, colon, ovarian, cervical and uterine origins. All were in either Stage III or Stage IV the cancers had metastased widely either to lung, liver, bladder or rectum).All had been treated with either radiation or chemotherapy after surgery but experienced no positive results. The bromelainase were administered in doses of 20-30 mg/kg (75 to 100 GDU/day) based on 50-60 kg of body weight. That is 1500 mg-to-2000 mg/day (or 5,000 to 6,000 GDU/day) and Quercetin in an amount of 2 mg/kg/day divided into two doses. Patients were monitored with bi-weekly blood tests consisting complete blood counts, chemistry-7, chemistry-24, kidney and liver function tests, tumor markers, coagulation factors; and X-ray or CT scan in appropriate areas to determine the size of the tumors. There were no abnormalities in all the blood tests, no anemia, no leucopenia, no thrombocytopenia, no abnormal kidney nor liver function tests. Tumor markers decreased, tumors were shrank and decreased in size on the x-ray or CT scan measurement. Patients' lifestyle become manageable, and improved considerably. The treatment period were varied from 6 to 12 months. At this report in the treatment groups no patients expired, however, all patients in control group, who had not wish to be treated, succumbed to their related cancers in 6-12 months.

(Step-A) After blood samples were collected and allowed to stand for 2 hours, then centrifuged at 4000 rpm for 10 minutes. (Megafuge, Baxter-Heraeus freeze before analysis. The chemistry analysis was carried out by blood chemistry analyzer (Cobra-Integra-700, Roche Diagnostic Lab. Indiana.) to determine the changes in total cholesterol, HDL, LDL, triglycerides, liver function tests (such as SGOT, SGPT, G-GPT), renal function tests, and coagulation factors (PT, PTT)(Bayer-MLA-Electra-900 Automatic coagulation Timer). All results were tested with student t-test and Microsoft Excel-7.0 program. The results are depicted in Table-III. Which were all within normal limits. Table-III, This table presents blood analysis of 24 volunteers suffering from various types of cancer with bromelain oral therapy. TC; (210.3+_30.2 mg/dl), TRG ; (165.5+_28.3 mg/dl), HDL; (43.3+_22.2 mg/dl), SGOT ; (34.7+_6.2 u/l), SGPT ; (63.3+_5.6 u/l), GGTP; (7.2+_2.1 u/l)WBC; (6.7+_2.8 k/ul), Hb; (12.3+_2.1 gm/di) Therefore, we concluded that the treatment of these various represented types of cancers with large doses and prolong periods with BROMELAIN are effective and without side effects.

### EXAMPLE 5

### Bromelain (Comosain) Intravenous Administration to Inhibit Tumor Growth in Humans

In addition to a control group of two untreated individuals, tested groups were established as follows; twelve human volunteers age from 4th through 6 decades, were divided into six groups of two, all of whom m were in Stage -3, ovary, cervix, and uterus. In the past, all patients had chemotherapy, and/or radiation therapy after surgery without effect. They were given Bromelain (Comosain) in doses of 50 GDU/kg (20 mg/kg) body weight of 50 to 60 kg, which equivalent of 1000 mg each week/(500 mg twice a week), Bromelain was administered intravenously in 6-8 hours period, for the 3 to 6 months. In addition to the Intravenous infusion of bromelain, all patients in the test groups also received oral bromelain in doses of 50 mg/kg/day (3000 mg/day) to increase therapeutic effects. Blood tests, including CBC, Chemistry-7, and 24, liver and renal function tests, lipid profile, coagulation factors, PT., PTT, IGG, tumor marks were tested every 6 weeks. And x-ray, and CT scan were performed every 3-4 months to measure the size of the tumors. The blood analysis on these groups of patients showed not affects from the bromelain infusion treatment. (The results are not depicted.) At the conclusion of the 6 months treatment period, the results are depicted as follows:

(A) Two breast cancer patients
(1). One patient experienced left breast tumors shrinkage from 6.5 cm×6.4 cm to 2.5 cm×1.8 cm in size. The left axillary lymph node shrank from 2.5 cm×1.6 cm to 0.5 cm×0.4 cm (Left breast).with 3 months of intra-venous (IV) bromelain therapy, and achieved complete remission in 6 months
(2) The second patient's right breast tumor shrank from 6.5 cm×4.8 cm to 2.8 cm×1.5 cm. with 4 months of intravenous bromelain therapy. And achieved complete remission in 9 months.

(B) Two Lung cancer patients
(1) One patient experienced the shrinkage from 4.6 cm×5.1 cm to 1.8 cm×2 cm with 12 weeks of intra-venous bromelain therapy. With further therapy for 4 months the tumors were completely remission.
(2) Another patient with lung tumor of 3.9 cm×4.5 cm experienced reduction of the tumor to 1.5 cm×1.9 cm in 3 months treatment. with 5 more months of IV bromelain therapy, Patient achieved complete remission.

(C) Two Colon cancer patients
(1) Two patients with stage-IV disease and widely metastasis in the abdominal cavity, have been treated with intra-venous bromelain infusion for 6-9 months, and showed no evidence of persistent disease.

(D) Two Ovarian cancer patients
(1) Both patients suffered stage-III C with wide intra-abdominal metastasis, both had been treated with intra-venous bromelain infusion for 6 months, and showed no evidence of persistent disease on CT scan and Ultrasound examination.

(E). Two patients with cervical cancers
(1) Both patients were stage-IV with rectal and urinary bladder invasions. After intense bromelain intra-venous treatment for 9 months, there was no further evidence bladder or rectal invasion. No tumors could be detected.

(F). Two patients with Uterine cancer
(1) Both patients were Stage-III with intra-vaginal metastasis, After intra-venous bromelain treatment for 4-6 months, tumors in the vagina showed necrosis and fibrosis with no evidence of persistent disease

(G). Two patients comprising control group, who refused to be treated

(1) One patient with cervical cancer stage-IV with cervical spine metastasis, and one patient with breast cancer stage-IV with pulmonary metastasis. Both patients refused treatment, and both succumbed to their diseases with pulmonary, and septic infections in 9 to 12 months after frequent in-hospital cares and treatments.

(H) A case of Hepatoma (hepato-cellular carcinoma); this tumor when it was discovered it was measured 15 CM×15 CM×10 CM in size, the surgical resection was not feasible, he received one course of chemotherapy without result and suffered from sepsis and bacteremia for 2 months, then he was given P.O. Comosain at a dose of 3000 mg/day, and IV Bromelain therapy for 7 months, patient achieved complete remission.

Again, this is evidence that administration of Bromelain (Comosain) extract (Bromelainases) in humans could treat various types of cancer and neoplastic diseases. Liver and renal function, lipid profile, white blood cells and hemoglobin were not affected. There are no side effects and toxicities.

### EXAMPLE -6

### Pharmeutical Formulation and Preparation

Hard and/or soft gelatin capsules are prepared with ingredients as follows:

Formulation-1

Quantity (mg/capsule): Active ingredient (1) Comosain 500, Active ingredient (2) Quercetin 250, Ascorbic acid 200, Starch or Lactose (carrier) 50, Total 1000 mg.

Formulation-2

Quantity (mg/capsule): Active ingredient (1) and (2) 1000, Ascorbic acid 300, Starch or Lactose (carrier) 200, Total 1500 mg.

Formulation-3

Quantity (mg/capsule): Active ingredient (1) and (2) 1600, Ascorbic acid 300, Starch or Lactose (carrier) 100, Total 2000 mg.

Formulation-4

Quantity (mg/vial): Active ingredient-1; Comosain 1 gram, Active ingredient-2; Quercetin 250 mg, Ascorbic acid 1000 mg/cc, Normal saline solution 3.0 ml which constitutes total volume of 5 ml for injection.

Formulation-5

Quantity (mg/vial): Active ingredient-1; Comosain 2 gram, Active ingredient-2; Quercetin 500 mg, Ascorbic acid 1000 mg/cc, Normal saline solution 3.0 ml which constitutes total volume of 5 ml for injection.

Formulation -6

Quantity (mg/vial): Active ingredient-1; Comosain 3 gram, Active ingredient-2; Quercetin 750 mg, Ascorbic acid 1000 mg/cc, Normal saline solution 6.0 ml which constitutes total volume of 10 ml for injection.

While the present invention and discovery has been described with respect to the above specific descriptions and embodiments, it should be recognized that various modifications and changes may be made to the present invention by those skills in the art which also fall within the scope of the invention as defined by the appended claims and their legal equivalents

1). The present discovery and invention relates to the methods and compositions for treating and/or preventing various types of cancer in a mammal, which comprise of the administration thereto of an effective amount of Glyco-Polypeptides enzyme Complex of Comosain, Bromelainases, Ananase, Pepsin, Trypsin and Bio-Flavonoids of Quercetin, Rutin, Naringenin, Genistein, Hespertin, etc and/or a mixture thereof.

2). The method of claim 1: Wherein the mammal is human.

3). The method of claim 1: Wherein the Glycopolypeptides, such as Comosain, Bromelainases, Ananase, are derived from Anana Comosus & Pepsin and Trypsin are from Animal Gastrointestinal juice. Quercetin, Rutin, Genistein, Naringenin, Hesperetin, are derived from Fructus Crataegus and Citrus Fruits, and the mixture of above complex is administered in the form of pharmaceutical composition containing an effective amount of the above components and pharmaceutically acceptable excipients, carriers or diluents.

4). The method of claim 1: wherein the effective amount of the Comosain, Bromelain, Ananase, Pepsin, Trypsin, Quercetin, Rutin, Genistein, Naringenin, Hesperetin, and/or a mixture thereof ranges from 0.1 to 500 mg/kg/day of body weight.

5). The method of claim 1: the Comosain, Bromelainases, Ananase, Pepsin, Trypsin Quercetin, Rutin, Genistein, Naringenin, Hesperetin, complex and with a solvent selected from the group consisting of water, a low alcohol and an aqueous alkali- or alkaline earth-metal hydroxide solution and/or an acid solution.

6). The method of claim 3, wherein the Comosain, Bromelainase, Ananase, Pepsin, Trypsin, Quercetin, Rutin, Genistein, Naringenin, Hesperetin, complex have cytotoxic effect (through T-cells, and mononuclear cells), anti-metastatic effect, anti-platelet aggregation, anti-inflammation, and anti-tumor- genesis (anti- proliferation and tumor necrotizing factors=TNF) and effect against various types of cancer cell lines and bacteria in animal and human experiments both in vitro and in vivo, such as breast, colon, lung, ovarian, cervical, uterine cancer, and hepatocellular carcinoma, etc.

7). The method of claim 3, where in the antitumor, anti-bacteria, and anti-inflammation effects are through T cells, and mononuclear cells. Massive cascade production of Interleukins IIB, II6, II8, and TNFα from TCRS/CD-2, TCRS/CD-3 which induce lyses of surface antigens of CD-44, CD-44s, CD-44v, CD-45, CD-47 in bacteria and tumor cells. And Through the following mechanism and pathways:
(A) First signal is generated with antigen peptide presented by the Major Histocompatibility Complex (MHC) expressed on Antigen Presenting Cells (APCs).
(B) The second stimulatory signal is generated by ligation of CD28 receptors on T-cells with B-7 family of ligands on APC.
(C) A key element in the signaling pathway involved in transducing receptor-initiated signals to the nucleus is the Major Mitogen Activating Protein Kinases, (MMAPK) and through Extracellular Signal-Regulated Protein Kinase of ERK-1 and ERK-2., ERKs are serine/threonine kinase (TPK, Tyrosine phosphorylation kinase) that are activated when phosphorylated on Tyrosine and Threonine residues. There are two other members of MMAPK that is C-JUN (NH) 2, (JNKs) which also require phosphorylation for activation. All the above events of signaling require Tyrosine Phosphorylation as inhibitors of Protein Tyrosine Kinase (PTKS) inhibit many events associated T-Cell receptors (TCRS) activation and Interleukin IIB, II-6, II-8) and TNFs (Tumor Necrotizing Factors) massive production.

8). Assembly of expression vector for the Comosain's minigene and preparation of genetically modified organism (GMO). For direct expression of the genomic Comosain's gene, 4.8 kilobase (kb) of BstTy/Se- and BamAs/GL fragment of Comosain, which contains the entire gene. after converting the BstTy/Se (tyrosine-serine 10 amino acids)site into Bst As/GL(asparagine-to-glycine 20 amino acids) site with a synthetic linker(pBR322 ori) the fragment was insert into the unique BamAs/GL site of the expression vector pDSVL, which contains a dihydrofolate reductase (DHFR) minigene. The resulting Plasmid DSVL-gPlCOS (gene Plant Comosain) was then used to transfect New Zealand white rabbit ovarian (NWRO) cells by the calcium phosphate microprecipitate method. The transformants were selected by the medium lacking hypoxanthine and thymidine. The culture medium used was Dulbecco's modified Eagle's medium supplemented with 10% fetal calf serum, penicillin, streptomycin, and glutamine.

9). Isolation of Comosain mRNA.: The 4.8 kilobase Bst ty/se-Bam As/GL restriction fragment from Comosain was inserted into shuttle vector, pSV4ST. The resulting chimeric plasmid pSV gPLComo was used to transfect Cos-1 cells by the calcium phosphate micoprecipitate method. After culture for 72 hrs, RNA was prepared from the transected cells by the guanidinium thiocyanate procedure of Chirgwin et al. and poly (A)+ mRNA was isolated by binding to oligo-cellulose.(Aviv & Leder).

10). cDNA cloning.: A Comosain cDNA bank was constructed according to a modification of the general procedures of Okayama and Berg by using the poly(A)+ mRNA described above (Mol. cell biology 2, 161-170, 1982).

11). DNA sequencing.: Restriction fragments were cloned into M 13 phage vectors by using Escherichia coli strains JM 103 and/or JM 109 as host. (Messing, J. of methods enzymology 1983) and were sequenced by the dideoxy method of Sanger et al. some regions were sequenced by kinase labeling or end-fill labeling of restriction fragments followed by chemical cleavage as described by Maxam and Gilbert. (J. of methods of enzymology 1980).
985). The final Comosain Recombinants were collected, extracted, washed with ethyl alcohol, and purified by AKAT Prime (GE Co.) and/orFPLC-cation-exchange chromatography, to produce of F4, F5, (other bromelainases) and F-9a, F-9b (Ananase and Comosain) for future use.
